(19) United States
(12) Patent Application Publication
(10) Pub. No.: US 2024/0400644 A1
(43) Pub. Date: Dec. 5, 2024

(54) FUSION PROTEINS COMPRISING 071 CORE PEPTIDE AND USE THEREOF

(71) Applicant: Acroimmune Guangzhou Biotech Ltd, Guangzhou (CN)

(72) Inventors: Qunmin ZHOU, Guangzhou (CN); Xianfeng FANG, Guangzhou (CN); Dongling LI, Guangzhou (CN); Libing MU, Guangzhou (CN)

(73) Assignee: Acroimmune Guangzhou Biotech Ltd, Guangzhou (CN)

(21) Appl. No.: 18/696,219

(22) PCT Filed: Sep. 27, 2022
(86) PCT No.: PCT/CN2022/121516

§ 371 Date: Mar. 27, 2024

(30) Foreign Application Priority Data
Sep. 28, 2021 (WO) PCT/CN2021/121237

(51) Int. Cl. C07K 14/705; A61K 38/00

(52) U.S. Cl. CPC C07K 14/70596; A61K 38/00; C07K 2319/30; C07K 2319/91

(57) ABSTRACT

An isolated 071-core-fragment, where an amino acid sequence of said isolated 071-core-fragment is as set forth in SEQ ID NO: 01. A protein, including a 071 core-derived-region, where said 071 core-derived-region consists of a single copy of a 071-core-fragment; or two or more copies of 071-core-fragments directly or indirectly linked to each other.

### BACKGROUND OF THE INVENTION

Inflammation is an innate immune response to foreign pathogen infection and self-tissue injury. The inducers of inflammation thus can be classified into two categories. The first and the perhaps more potent one is so called as pathogen-associated molecular pattern (PAMP), and the second and less studied one is so called as damage (danger)-associated molecular pattern (DAMP) (Janeway C A. Cold Spring Harbor Symposia on Quantitative Biology. 1989; 54:1; Matzinger P. Annual Review of Immunology. 1994; 12:991). PAMP present on almost all microbial pathogens, and the survival of multi-cellular organisms is dependent on their ability to recognize these PAMP in invading microbial pathogens and to induce immune response or defense reactions. Some examples of the well characterized PAMPs are Lipopolysaccharide (LPS), poly(I:C), Pam3Cys, CpG DNA and etc. Toll-like receptors, members of an evolutionarily ancient family, plays a crucial role in the detection of PAMPs in microbial infection and the induction of immune and inflammatory responses. (Medzhitov R, Preston-Hurlburt P, Janeway C A Jr. A human homologue of the Drosophila Toll protein signals activation of adaptive immunity. Nature. 1997; 388:394; Janeway C A Jr, Medzhitov R. Innate immune recognition. Annu Rev Immunol. 2002; 20:197-216; Medzhitov R, Janeway C A Jr. The Toll receptor family and microbial recognition. Trends Microbiol. 2000; 8:452-6.).

Unlike PAMP, which are present only on invading microbial pathogens, DAMP by nature, are host self-components which are released by necrotic or damage cells/organs when they are under stress or face microbial invasion. Some of the well characterized DAMP including the heat-shock proteins (HSP70, HSP90), cellular DNA/RNA and high mobility group box-1 (HMGB1). These DAMPs can be either actively secreted by immune-response cells or passively released by necrotic or damage cells into extracellular environment. For example, HMGB1, normally a nuclear located chromatin-binding protein, can be either actively released by innate immune cells upon their exposure to exogenous pathogen-derived molecules, or passively released by ischemia or cell injury in the absence of pathogen invasion. The released HMGB1 is then detected by immune systems by binding to its partners such as TLRs, RAGE, Tim-3, or GPI-anchored siaoglycoprotein CD24 and then triggers a typical non-pathogen induced inflammatory responses or sterile inflammation.

Recent data further support a role for sialoside-based pattern recognition by members of the Siglec family to attenuate innate immunity. (Liu Y, Chen G Y and Zheng P: Sialoside-based pattern recognitions discriminating infections from tissue injuries. Curr Opin Immunol. 2011:41-5). Since CD24-Siglec 10/G interaction selectively dampens host response to DAMPs but not PAMPs, this sialoside-based pattern recognition may serve as a foundation to discriminate PAMP from DAMP. Molecules such as nature occurred CD24 glycoprotein or genetically engineering fusion protein such as CD24Fc bind to members of Siglec family and diminish the overall inflammatory response induced by DAMP and found to have therapeutic effects in a number of diseases models including graft vs host diseases (GVHD), rheumatoid arthritis, and pathological setting in which infections cause tissue injuries such as COVID-19, influenza pneumonia and sepsis. Nevertheless, there is still an urgent need to develop a safe and more potent biological product with enhanced anti-inflammation properties.

### SUMMARY OF THE INVENTION

The present disclosure provides compositions of a glycosylated or sialylated core peptide (named as 071 core) and compositions of proteins based on fusion of one or more copy of the peptide to the Fc fragment of human immunoglobulin and their use in treating diseases propagated by inflammations associated with tissue injuries.

The present disclosure provides an isolated 071-core-fragment, wherein the amino acid sequence of said 071-core-fragment consists of the amino acid sequence as set forth in SEQ ID NO: 01.

The present disclosure provides a protein comprising a 071 core-derived-region, said 071 core-derived-region consists of: single copy of 071-core-fragment; or two or more copies of 071-core-fragments directly or indirectly linked to each other; the amino acid sequence of said 071-core-fragment consists of the amino acid sequence as set forth in SEQ ID NO: 01.

The present disclosure provides an immunoconjugate, comprising the 071-core-fragment of the present disclosure, or the protein of the present disclosure.

The present disclosure provides a nucleic acid, encoding the 071-core-fragment of the present disclosure, or the protein of the present disclosure.

The present disclosure provides a vector, comprising the nucleic acid the present disclosure.

The present disclosure provides a cell, comprising and/or expressing the 071-core-fragment of the present disclosure, the protein of the present disclosure, the immunoconjugate the present disclosure, the nucleic acid the present disclosure, and/or the vector the present disclosure.

The present disclosure provides a composition, comprising the 071-core-fragment of the present disclosure, the protein of the present disclosure, the immunoconjugate the present disclosure, the nucleic acid the present disclosure, the vector the present disclosure, and/or the cell the present disclosure, and optionally a pharmaceutically acceptable carrier.

The present disclosure provides a method for preparing the 071-core-fragment of the present disclosure, or the protein of the present disclosure, comprising culturing the cell the present disclosure under a condition enabling the expression of said 071-core-fragment or said protein.

The present disclosure provides a method for regulating a Siglec related signaling, comprising administering to a subject in need thereof an effective amount of the 071-core-fragment of the present disclosure, the protein of the present disclosure, the immunoconjugate the present disclosure, the nucleic acid the present disclosure, the vector the present disclosure, the cell the present disclosure, and/or the composition the present disclosure.

The present disclosure provides a method for regulating an immune response, comprising administering to a subject in need thereof an effective amount of the 071-core-fragment of the present disclosure, the protein of the present disclosure, the immunoconjugate the present disclosure, the nucleic acid the present disclosure, the vector the present disclosure, the cell the present disclosure, and/or the composition the present disclosure.

The present disclosure provides a method for repressing an immune-mediated tissue damage mediated by danger-associated molecular patterns (DAMPs), comprising administering to a subject in need thereof an effective amount of the 071-core-fragment of the present disclosure, the protein of the present disclosure, the immunoconjugate the present disclosure, the nucleic acid the present disclosure, the vector the present disclosure, the cell the present disclosure, and/or the composition the present disclosure.

The present disclosure provides a method for preventing, ameliorating and/or treating a disease or condition caused by an inflammatory response arising from tissue injuries from infectious agents, comprising administering to a subject in need thereof an effective amount of the 071-core-fragment of the present disclosure, the protein of the present disclosure, the immunoconjugate the present disclosure, the nucleic acid the present disclosure, the vector the present disclosure, the cell the present disclosure, and/or the composition the present disclosure.

The present disclosure provides a method for preventing, ameliorating and/or treating a disease or condition caused by acute tissue damage from wound, comprising administering to a subject in need thereof an effective amount of the 071-core-fragment of the present disclosure, the protein of the present disclosure, the immunoconjugate the present disclosure, the nucleic acid the present disclosure, the vector the present disclosure, the cell the present disclosure, and/or the composition the present disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCES

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 Shows the schematic structure of the 071 fusion proteins, comprising one or more copies of 071 core tandem repeats and IgG-Fc tail. The preferred formation of 071 Fc-fusion protein is a dimer, covalently linked via disulfate chains of the hinge region and non-covalent interactions between CH2 and CH3 domains of human IgG1.

FIGS. 2A-2B SDS-PAGE gel electrophoresis and SEC-HPLC analysis of purified AI-071 Fc-fusion protein. FIG. 2A shows the picture of a representative SDS-PAGE gel electrophoresis of purified AI-071 Fc-fusion protein. Two μg of purified AI-071 fusion protein, either in reducing (R) or non-reducing conditions (NR), was loaded into SDS-PAGE. After electrophoresis, the gel was stained with Coomassie Brilliant Blue dye. The molecule weight (kDa) of protein marker (M) in the gel was indicated to the left. FIG. 2B shows the separation profile of purified AI-071 protein by a size exclusion chromatography (SEC)-high performance liquid chromatography (HPLC) analysis.

FIGS. 3A-3C Show the binding to the plate-bound AI-071 or CD24Fc protein by different anti-CD24 mAbs in ELISA. AI-071, CD24Fc (CHO-cell derived), CD24Fc (HEK293 cell derived) or human IgG1-Fc control protein were dissolved in coating buffer at concentration of 10 μg/ml and added into 96-well plates (50 μL/well). After washing and blocking with PBS-0.1% Tween20 solution (PBST), 100 μl of 2-fold serial dilutions of either SN3 (ab 134375 from Abcam), ML5 (ab278509 from Abcam) or H3L3 antibodies were added into the plates. The bound antibodies were detected by biotin-labelled goat anti-mouse IgG-Fc antibodies followed by horseradish peroxidase (HRP)-labelled Avidin. The plates were then incubated with o-Phenylenediamine (OPD) substrates. The representative ELISA results are showed here. FIG. 3A shows the binding profile of AI-071 or CD24Fc to a two-fold serial dilution of the mouse anti-CD24 mAb SN3. The EC50 values of the binding are shown in the underneath table. FIG. 3B shows the binding profile of AI-071 or CD24Fc to a two-fold serial dilution of H3L3, a humanized anti-CD24 mAb. The EC50 values of the binding are shown in the underneath table. FIG. 3C shows the binding profile of AI-071 or CD24Fc to a two-fold serial dilution of ML5, another mouse anti-CD24 mAb. The EC50 values of the binding are shown in the underneath table.

FIGS. 3D and E show the binding of anti-CD24 mAb SN3 or ML5 to neuraminidase treated AI-071. AI-071 fusion protein (5 μg/mL) was incubated in 1×PBS buffer containing increasing concentration (0, 1, 5, 10, 20 and 250 mU/mL) of neuraminidases (Sigma, N2867) at 37° C. for 3 hours. After the treatment, the AI-071 protein was then added into 96-well plates (100 μL/well) and incubated at 4° C. overnight. After washing and blocking with PBST, the plates were incubated with 2-fold serial dilutions of either sialylation-dependent anti-CD24 mAb SN3 (starting concentration at 5 μg/mL) or sialylation-independent anti-CD24 mAb ML5 (starting concentration at 5 μg/mL) at room temperature for 1 hour. After washing, the plates were incubated with HRP-conjugated goat anti-mouse IgG-Fc antibodies (1:5000 dilution, SinoBio, Cat: SSA006) followed by the addition of o-Phenylenediamine (OPD) substrates. FIG. 3D shows the binding of SN3 mAb to the neuraminidase treated AI-071 fusion protein coated on the plate. FIG. 3E shows the binding of ML5 mAb to the neuraminidase treated AI-071 fusion protein coated on the plate.

FIG. 4A shows the binding of AI-071 protein or CD24Fc to plate-bound human Siglec-10 in ELISA. 96-well plates were coated (100 μL per well) with 0.2 μg/mL of HEK293 cell derived Siglec10-mlgG2aFc fusion protein (AcroBiosystems, SIO-H525b) at 4° C. overnight. After blocking with SuperBlock (Thermo, 37515) at room temperature for 1 hour, 100 μL of 2-fold serial dilutions of AI-071 or CD24Fc protein (all starting at 1.5 mg/mL) were added. The bound AI-071 or CD24Fc protein was then detected by HRP-labeled goat anti-human IgG-Fc antibody (1:5000, Invitrogen, A18829) followed by the addition of tetramethylbenzidine (TMB) substrates. The EC50 values of the binding are shown in the underneath table.

FIG. 4B-C shows the binding of AI-071 protein to other human siglec family members. FIG. 4B shows the binding of AI-071 protein to human Siglec-1, Siglec-2, Siglec-3, Siglec-4A, Siglec-5 and Siglec-6. FIG. 4C shows the binding of AI-071 protein to human Siglec-7, Siglec-8, Siglec-9, Siglec-11, Siglec-14 and Siglec-15. Briefly, 2-fold dilutions of biotin-labeled AI-071 fusion protein were added into 96-well plated coated with indicated Siglec-Fc fusion proteins (all coating at 0.2 μg/mL), the amount of bound AI-071 protein was then detected by adding Avidin-HRP and OPD.

FIG. 5A shows the binding of AI-071 protein or CD24Fc to human HMGB1 in ELISA. 96-well plates were coated (100 μL per well) with 0.1 μg/mL HEK293 cell derived HMGB1-His tag protein (AcroBiosystems, HM1-H5220) at 4° C. overnight. After blocking with SuperBlock (Thermo, 37515) at room temperature for 1 hour, 100 μL of 2-fold serial dilutions of AI-071 or CD24Fc (all starting at 1.5 mg/mL, dilution buffer: PBST-1% BSA solution containing 1 mM MgCl2 and 1 mM CaCl2) were added. The bound AI-071 or CD24Fc protein was then detected by adding HRP-labeled goat anti-human IgG-Fc antibody (1:1000, Invitrogen, A18829), followed by the addition of tetramethylbenzidin (TMB) substrates. The EC50 values of the binding are shown in the underneath table. FIG. 5B shows the association of AI-071 protein with human HMGB1 in a pull-down assay. Recombinant HMGB1-His protein sample was incubated with AI-071, human IgG-Fc, or none for 5 min, the mixture was then incubated with protein A-conjugated beads to capture (or pull-down) the bound proteins. The captured proteins were then separated in a SDS-PAGE gel and visualized by Coomassie Brilliant Blue dye staining. The left panel shows the input samples, and the right panel shows the pulldown samples, as marked. As indicated on the top of each gel, lane 1 represents the sample containing HMGB1 only, lane 2 represents the sample containing HMGB1 and human IgG-Fc, and lane 3 represents the sample containing HMGB1 and AI-071, lane M is the protein molecule weight marker sample. The positions of AI-071 protein, HMGB1 protein in input or pulldown sample are indicated to the left side of each gel, whereas the molecule weight (kDa) of protein marker is displayed to the right side of each gel.

FIGS. 6A-6D illustrate the therapeutic effects of AI-071 to DSS-induced inflammatory bowel disease in mice. FIG. 6A illustrates the procedure of DSS-induced mouse inflammatory bowel disease and the treatment schedule. The AI-071 protein or vehicle control was administrated to model mice by i.p injection on day 0 and day 6. Model mice were then observed daily, with body weight recorded, and survival rate calculated until day 14. FIG. 6B illustrates the animal body change (gram) vs time (day) curve in AI-071 protein treated group or vehicle control treated group. FIG. 6C illustrates the body weight loss rate (%) vs time (day) curve in AI-071 protein treated group or vehicle control treated group. FIG. 6D illustrates the animal survival rate (%) vs time (day) curve in AI-071 protein treated group (n=10) or vehicle control treated group (n=10).

FIGS. 7A-7B illustrate the mouse model to test the therapeutic effects AI-071 to LPS-induced airway/lung inflammation. FIG. 7A illustrates the procedure of LPS-induced mouse airway/lung inflammation. Briefly, 200 μg LPS in 40 μl saline was administered intranasally (i.n.) to the mice on day 0, and day 1. AI-071 (20 mg/kg) or vehicle control (buffer) were administered by i.v. at one hour after LPS treatment on Day 0. On Day 3 or Day 4, bronchoalveolar lavage fluids (BALF) were harvested and the total cell account, neutrophil cell number, level of cytokines and total protein in BALF were counted or measured. Lung tissues were also collected and processed for haematoxylin and eosin (H&E) staining. FIG. 7B illustrates the treatment groups for the LPS-induced lung Inflammation study in mice.

FIGS. 8A-D show the effects of AI-071 treatment in the LPS-induced mouse airway inflammation model. FIG. 8A illustrates the number of leukocytes, FIG. 8B illustrates the total neutrophil counts, FIG. 8 C illustrates the percentage of neutrophils in CD45+ cells, and FIG. 8D illustrates the protein concentrations in BALF samples collected from Day 3 or Day 4 after LPS inhalation.

FIGS. 9A-D illustrate the lung histopathological and damage score assessed on day 3 or day 4 after LPS inhalation from different treatment group mice. FIG. 9A-C show the representative images of the H&E stained sections of lung tissues from either saline+buffer (A), LPS+buffer (B), or LPS+AI-071 (C) treated mice. FIG. 9D shows the lung histopathological damage score assessed on Day 3 and Day 4 after LPS inhalation (Data are mean±SD, one-tailed unpaired t-test).

FIGS. 10A-C illustrate the levels of major cytokines in BALF samples harvested on Day 3 or Day 4 after LPS inhalation from different treatment group mice. FIG. 10A shows the levels of TNF-α, FIG. 10B shows the level of IL-6, and FIG. 10C shows the level of IL-1B. Cytokines in BALF were measured by LEGENDplex™ multi-analyte flow assay kit (BioLegend). Results shown here were normalized by Log10 transformation (Data are mean±SD) and analyzed by one-tailed unpaired t-test.

FIGS. 11A-B show the concentrations of AI-017 detected in the BALF or blood plasma samples collected from LPS-induced mice after the treatment. FIG. 11A shows the concentration of AI-071 in BALF samples collected on day 3 or day 4. FIG. 11B shows the concentration of AI-071 in plasma samples collected on day 4.

FIGS. 12A-12B illustrate the testing method and the activities of AI-071 to collagen antibody induced arthritis (CAIA). FIG. 12A illustrates the methods of inducing CAIA model and the treatment schedule. Arthritis was induced by i.v. injecting mice with anti-collagen cocktail antibodies (a mixture of 5-clones, 1.5 mg/mouse) on day 0, and followed by i.v. injecting 50 μg LPS on day 3 and day 4. Mice were then randomly separated into two groups, receiving either AI-071 protein (50 mg/kg) or vehicle control on day 0. On day 14, each mouse was re-administered 0.8 mg of anti-collagen cocktail mAbs by i.v. injection, followed by i.p. injection of 35 μg LPS on day 16. On day 19, these mice were treated with either a second dose (1 mg) of AI-071 or saline control. Mice are monitored daily, with body weight recorded, disease scored and survival rate calculated up to day 30. FIG. 12B illustrates the disease score ratio (day/day 19) change from starting from day 19 to day 30 in animal group treated with either AI-071 protein (n=10) or vehicle control (n-10).

FIG. 13 shows the amino acid sequences of mature CD24 core protein and the locations of three peptide fragments corresponding to different regions of CD24. The top shows the amino acid sequences of the mature CD24 peptide backbone with indicated 16 potential O-linked glycosylation sites (marked by the symbol [symbol] on the top of Ser and Thr residues) and 2 potential N-linked glycosylation sites (marked by the symbol [symbol] on the top of Asn residues); the 12 amino acid long sequence highlighted in the middle region of CD24 represents the 071-core peptide. The peptide region or potential amino acid sequence (epitope) recognized by ML5 mAb or SN3 mAb was also indicated on the top of CD24 peptide backbone. The boxed regions underneath the CD24 peptide backbone represent three different fragments (namely as 071-X, 071-Y and 071-Z) used for the construct of different versions of AI-071-Fc protein. The amino acid sequences highlighted in 071-Y or 071-Z peptide represent those overlapped with 071-core peptide.

FIG. 14 shows the amino acid sequences of three different peptide fragments derived from CD24. Like 071-core peptide, each of these three peptides (071-X, 071-Y and 071-Z) also is 12 amino-acid long (12mer). The amino acid sequences highlighted in 071-Y and 071-Z represent those overlapped with 071 core peptide.

FIGS. 15A-15B show the gel pictures of SDS-PAGE analysis of purified different version of AI-07-Fc protein or control Fc-protein. FIG. 15A shows the SDS-PAGE gel of the purified different version of AI-07-Fc protein or control Fc-protein in non-reducing (NR) conditions. FIG. 15B shows SDS-PAGE gel of the purified different version of AI-07-Fc protein or control Fc-protein in reducing (R) conditions. About 5 μg of purified fusion protein of each sample in DTT-reducing (R) or non-reducing conditions (NR), was loaded into SDS-PAGE gel. After electrophoresis, the gel was stained with Coomassie Brilliant Blue dye to visualize the protein bands. The molecule weight (kDa) of protein marker (M) in the gel was indicated to the right.

FIGS. 16A-16B show the binding of the different versions of AI-07-Fc protein or Fc-control protein to anti-CD24 mAb SN3 in ELISA. FIG. 16A shows the binding of 2-fold serial dilutions of purified, CHO-cell derived AI-071, AI-071-X, AI-071-Y, AI-071-Z, AI-071-1 copy, AI-071-2 copies or human IgG1-Fc control protein samples (starting concentration all at 25 μg/mL) to SN3 mAb coated in 96-well plates (coating concentration at 2 μg/mL). The amount of SN3-captured protein was then detected by HRP-conjugated goat anti human IgG Fc (Fc-specific) antibodies (Sigma, A0170) followed by the addition of O-Phenylenediamine (OPD) substrates. FIG. 16B shows the binding of 2-fold serial dilutions of anti-CD24 mAb SN3 (starting concentration at 10 μg/mL) to AI-071, AI-071-X, AI-071-Y, AI-071-Z, AI-071-1copy, AI-071-2copies, CD24Fc or human IgG1-Fc control protein coated on 96-well plates (coating concentration all at 10 g/mL). The amount of SN3 bound to the plate was detected by HRP-conjugated goat anti-mouse IgG Fc (SinoBio, Cat: SSA006, 1:5000 dilution) followed by the addition of O-Phenylenediamine (OPD) substrates.

FIGS. 17A-17B show the binding of the different versions of AI-07-Fc protein or Fc-control protein to anti-CD24 mAb ML5 in ELISA. FIG. 17A shows the binding of 2-fold serial dilutions of purified, CHO-cell derived AI-071, AI-071-X, AI-071-Y, AI-071-Z, AI-071-1copy, AI-071-2copies or human IgG1-Fc control protein samples (starting concentration all at 2 μg/mL) to ML5 mAb coated on 96-well plates (coating concentration at 2 μg/mL). The amount of ML5 captured protein was detected by HRP-conjugated goat anti human IgG Fc (Fc-specific) antibodies (Sigma, A0170, 1:2000 dilution) followed by the addition of O-Phenylenediamine (OPD) substrates. FIG. 17B shows the binding of 2-fold serial dilutions of anti-CD24 mAb ML5 (starting concentration at 10 μg/mL) to AI-071, AI-071-X, AI-071-Y, AI-071-Z, AI-071-1copy, AI-071-2copies, CD24Fc or human IgG1-Fc control protein coated on 96-well plates (coating concentration all at 10 μg/mL). The amount of ML5 bound to the plate was detected by HRP-conjugated goat anti-mouse IgG Fc (SinoBio, Cat: SSA006, 1:5000 dilution) followed by the addition of O-Phenylenediamine (OPD) substrates.

FIGS. 18A-18B show the binding of the different version of AI-07-Fc protein or control Fc-protein to anti-human IgG-Fc antibody in direct ELISA. 96-well plates were pre-coated with purified, CHO-cell derived AI-071, AI-071-X, AI-071-Y, AI-071-Z, AI-071-1copy, AI-071-2copies, CD24Fc or human IgG1-Fc control protein (coating concentration all at 1 μg/mL in FIG. 18A, and at 5 μg/mL in FIG. 18B). After blocking with 2% BSA in PBS, 2-fold serial dilutions of HRP-conjugated goat anti-human IgG-Fc (Fc-specific) antibodies (Sigma, A0170, staring concentration at 1:2000 dilution) were added into the plate. After washing, the amount of the HRP-conjugated anti-human IgG-Fc antibody bound to the plate was detected by adding O-Phenylenediamine (OPD) substrates.

FIG. 19A-19B show the binding of 2 different versions of AI-071-Fc protein containing either 1 copy of 071core (AI-071-1copy) or 2 copies of 071core (AI-071-2copies) to human Siglec-10 or HMGB1 in ELISA. A control sample of AI-071 fusion protein was included in the assay and served for comparison. FIG. 19A shows the binding of these 2 different versions of AI-071-Fc protein (AI-071-1copy, or AI-071-2copies) and control sample of AI-071 to GST-Siglec 10 protein. Briefly, a 96-well plate was coated with recombinant GST-Siglec-10 fusion protein (at concentration of 200 ng/ml). After washing and blocking, the plate was incubated with a two-fold serial dilution of different versions of AI-071-Fc protein or AI-071 control sample (all starting at 1.5 mg/mL), the bound protein was then detected by adding HRP-labeled goat anti-human (1:1000, Invitrogen, A18829), followed by the addition of tetramethylbenzidin (TMB) substrates. FIG. 19B shows the binding of these 2 different versions of AI-071-Fc protein (AI-071-1copy, or AI-071-2copies) and AI-071 control sample to his-tagged HMGB1 protein pre-bound to the plate, with AI-071 protein serves as control sample for comparison. Briefly, a 96-well plate was coated with 0.1 μg/mL HMGB1-His tag protein (AcroBiosystems, HM1-H5220, HEK293 cell derived). The plate was then incubated with 2-fold serial dilutions of different version of AI-07-Fc protein (all starting at 1.5 mg/mL, in PBST-1% BSA solution containing 1 mM MgCl2 and 1 mM CaCl2). The bound protein was then detected by adding HRP-labeled goat anti-human IgG-Fc antibody (1:1000, Invitrogen, A18829), followed by the addition of tetramethylbenzidin (TMB) substrates.

FIG. 20A-20B show the binding of 3 different other versions of AI-071-Fc protein (AI-071-X, AI-71-Y and AI-071-Z), to human Siglec-10 or HMGB1 in ELISA. Each of these three AI-071-Fc protein variants (AI-071-X, AI-71-Y and AI-071-Z) containing 5 copies of a 12 AA-long peptide derived from different portions of CD24 other than 071core peptide. Same as in FIG. 19, AI-071 fusion protein was included in the assay and served as a control sample for comparison. FIG. 20A shows the binding of these three different versions of proteins or AI-071 control sample to recombinant GST-Siglec-10 protein pre-coated on the plate. The ELISA method and sample preparation are same as that in FIG. 19A. FIG. 20B shows the binding of these three different versions of proteins or AI-071 control sample to recombinant HMGB1-His tag protein pre-coated on the plate. The ELISA method and sample preparation are same as that in FIG. 19B.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “peptide” or “polypeptide” may refer to a linked sequence of amino acids and may be natural, synthetic, or a modification or combination of natural and synthetic.

The term “glycopeptide” or “glycoprotein” may refer to a modification of natural or synthetic peptide or protein with sugar or oligosaccharide attached or linked to the amino acid residues.

The term “Substantially identical” may refer to a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, may refer to preventing, suppressing, repressing, or completely eliminating the disease. Preventing the disease may involve administering a composition of the present invention to an animal prior to onset of the disease. Suppressing the disease may involve administering a composition of the present invention to an animal after induction of the disease but before its clinical appearance. Repressing the disease may involve administering a composition of the present invention to an animal after clinical appearance of the disease.

A “variant” may refer to a peptide or polypeptide that differs in amino acid sequence by the insertion, deletion, or conservative substitution of amino acids, but retain at least one biological activity. Representative examples of “biological activity” may include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) may be recognized in the art as typically involving a minor change. These minor changes can be identified, in part, by considering the hydropathic index of amino acids, as understood in the art. The hydropathic index of an amino acid may be based on a consideration of its hydrophobicity and charge. It may be known in the art that amino acids of similar hydropathic indexes can be substituted and still retain protein function. In one aspect, amino acids having hydropathic indexes of +2 may be substituted. The hydrophilicity of amino acids can also be used to reveal substitutions that would result in proteins retaining biological function. A consideration of the hydrophilicity of amino acids in the context of a peptide may permit calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids may be influenced by the particular side chain of that amino acid. Consistent with that observation, amino acid substitutions that are compatible with biological function may be understood to depend on the relative similarity of the amino acids, and particularly the side chains of those amino acids, as revealed by the hydrophobicity, hydrophilicity, charge, size, and other properties.

The term “CD24” may refer to a protein or peptide. As used herein, CD24 may be a glycosylphosphatidylinositol (GPI)-anchored protein with potential O- and N-glycosylation sites. CD24 may encompasses CD24 proteins, protein fragments, protein analogs, oligopeptides, and/or a variant thereof. For example, the CD24 fragment may not include the full length CD24 protein. The UniProt No. for CD24 may be P25063.

The term “fusion” as used herein refers to a fused molecule, wherein the components of the fusion molecule may be linked to each other by bonds, like peptide bonds, either directly or via a peptide linker. The individual peptide chains of the fusion molecule may be linked non-covalently, for example by disulfide bonds.

### 071 Core or Fragment

In the present disclosure, a 12 amino acid long peptide was named as 071 core or fragment with its amino acid sequence STSNSGLAPNPT (SEQ ID NO: 01) disclosed here. This 12-amino-acid long peptide contains 5 potential mucin-like O-glycosylation sites (serine or threonine in STP motif) and may be heavily glycosylated or sialylated when attached to other partner proteins such as Fc of human IgG and expressed in mammalian cells. Repeats of this 12-amino-acid long peptide such as −2 (SEQ ID NO: 02), −3 (SEQ ID NO: 03), −4 (SEQ ID NO: 04), −5 (SEQ ID NO: 05) or more copies of it may have even greater O-glycosylation.

In the present disclosure, some variants of this 12 amino acid sequence STSNSGLAPNPT (SEQ ID NO: 01) are also provided. These variants may have one of the following amino-acid sequences: SASNSGLAPNPT (SEQ ID NO: 06); STSNSGLAPNPA (SEQ ID NO: 07); SASNSGLAPNPA (SEQ ID NO: 08). These variants may contain 3 or 4 mucin-like O-glycosylation sites and might be still heavily glycosylated or sialylated when attached to other partner proteins such as Fc of human IgG and expressed in mammalian cells. Repeats of this 12-amino-acid long peptide such as 2, 3, 4, 5, or more copies of it may have even greater O-glycosylation.

In the present disclosure, another type variant of this 12 amino acid sequence STSNSGLAPNPT (SEQ ID NO: 01) are also provided. For example, these variants may have one of the following amino-acid sequences: XTSNSGLAPNPT (X=S or T, SEQ ID NO: 9); SXSNSGLAPNPA (X=S or T, SEQ ID NO: 10); STXNSGLAPNPT (X=S or T, SEQ ID NO: 11); STSNXGLAPNPT (X=S or T, SEQ ID NO: 12); STSNTGLAPNPX (X=S or T, SEQ ID NO: 13); XTSNTGLAPNPX (X=S or T, SEQ ID NO: 14); STSNTXXAPNPT (X=S or T, SEQ ID NO: 15). For example, these variants may have one of the following amino-acid sequences: XXXNXGLAPNPX (each X is independently=S or T). These variants may contain 5, 6 or 7 mucin-like O-glycosylation sites and might be heavily glycosylated or sialylated when attached to other partner proteins such as Fc of human IgG and expressed in mammalian cells. Repeats of this 12-amino-acid long peptide such as 2, 3, 4, 5 or more copies of it may have even greater O-glycosylations. Wherein said the core region is 80% identical to 071 core. Wherein said the core region is 80%, 85%, 90%, 91%, 92%, 93%, 94%, 95%, 96%, 97%, 98%, or 99% identical to 071 core.

In the present disclosure, variants of the 12 amino acid sequence STSNSGLAPNPT (SEQ ID NO: 01) from other mammalian species are also provided. For example, these variants may have one of the following amino-acid sequences: STSNSGFAPNPT (SEQ ID NO: 16) from chimpanzee (Gorilla gorilla or Pan troglodytes); SSQSTSAAPSPA (SEQ ID NO: 17) from marmoset monkey (Callithrix jacchus); SSQNTSTTPNPA (SEQ ID NO: 18) from Cynomolgus monkey (Macaca fascicularis); GNQNISASPNPT (SEQ ID NO: 19) from mouse; GNQSISAAPNPT (SEQ ID NO: 20) from rat; SSQSTSTAPNPA (SEQ ID NO: 21) from Dog (Canis lupus familiaris) CD24. These variants may contain 3, 5 or 6 mucin-like O-glycosylation sites and might be heavily glycosylated or sialyated when attached to other partner proteins such as Fc of human IgG and expressed in mammalian cells. Repeats of this 12-amino-acid long peptide such as 2, 3, 4, 5 or more copies of it may have even greater O-glycosylation.

In this patent application, a series of 071 fusion proteins were created in a such way that multiple copies of the amino acid sequences of STSNSGLAPNPT (SEQ ID NO: 01) may be tandem linked together and may be fused with human IgG-Fc (hinge-CH2-CH3) with the amino acid sequences such as in SEQ ID NO: 28. The Fc-hinge region may also have one of the amino acid sequences as set in SEQ ID NO: 22, SEQ ID NO: 23, SEQ ID NO: 24, SEQ ID NO: 25, SEQ ID NO: 26, or SEQ ID NO: 27.

The present disclosure provides an isolated 071-core-fragment, wherein the amino acid sequence of said 071-core-fragment may consist of the amino acid sequence as set forth in SEQ ID NO: 01.

The present disclosure provides a protein comprising a 071 core-derived-region, said 071 core-derived-region may consist of: Single copy of 071-core-fragment; or two or more copies of 071-core-fragments directly or indirectly linked to each other; the amino acid sequence of said 071-core-fragment may consist of the amino acid sequence as set forth in SEQ ID NO: 01. The protein of the present disclosure may not comprise the fragment of other part of the fragment derived from the CD24.

For one example of the protein, wherein said two or more 071-core-fragments may comprise 2, 3, 4, 5 or more of said 071-core-fragments.

For one example of the protein, wherein at least two of said two or more 071-core-fragments may be directly linked to each other. For example, the 071-core-fragments may be independently directly linked or indirectly linked to each other. For example, two 071-core-fragments may be directly linked, and these two 071-core-fragments may be indirectly linked to another 071-core-fragment. For example, the directly linked may refers to two or more fragments may be linked by bonds, like peptide bonds. For example, the indirectly linked may refers to two or more fragments may be linked by peptide linker, like GnS linker.

For one example of the protein, wherein at least two of said two or more 071-core-fragments may be indirectly linked to each other via a linker. For one example of the protein, wherein said linker may be a peptide linker. For example, the peptide linker may be a (GnS) n linker such as GGGGS, or GGGGSGGGGGGGGS.

For one example of the protein, wherein said 071 core-derived-region may consist of the amino acid sequence as set forth in SEQ ID NO: 01, SEQ ID NO: 06, SEQ ID NO: 07, SEQ ID NO: 08, SEQ ID NO: 09, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21.

For one example of the protein, further comprising a second portion, said second portion may comprise a half-life extending portion. For one example of the protein, wherein said half-life extending portion may comprise an immunoglobulin fragment.

For one example of the protein, wherein said immunoglobulin fragment may comprise a Fc portion of said immunoglobulin. For one example of the protein, further comprising a second portion, said second portion may comprise an immunoglobulin fragment. For one example of the protein, wherein said immunoglobulin fragment may comprise a Fc portion of said immunoglobulin.

For one example of the protein, wherein said immunoglobulin fragment may comprise a hinge region of said immunoglobulin. For one example of the protein, wherein said immunoglobulin fragment may comprise a CH2 domain. For one example of the protein, wherein said immunoglobulin fragment may comprise a CH3 domain. For one example of the protein, wherein said immunoglobulin fragment may comprise a CH4 domain. For example, said immunoglobulin fragment may comprise hinge region and CH2 and CH3 domains of said Ig protein. For example, said Ig may be selected from the group consisting of IgG1, IgG2, IgG3, IgG4, and IgA. For example, said immunoglobulin fragment may comprise hinge region and CH3 and CH4 domains of said Ig protein. For example, said Ig may be IgM. For example, said immunoglobulin fragment may comprise hinge region and CH2, CH3 and CH4 domains of said Ig protein. For one example of the protein, wherein said immunoglobulin may be selected from the group consisting of IgG1, IgG2, IgG3, IgG4, IgM and IgA.

For one example of the protein, wherein said second portion may be directly or indirectly linked to said 071 core-derived-region.

For one example of the protein, wherein said second portion may be indirectly linked to said 071 core-derived-region via a linker. For example, said second portion may be directly linked to said 071 core-derived-region. For example, said second portion may be directly linked to said 071 core-derived-region, and said second portion may not comprise hinge region. For example, said second portion may be directly linked to said 071 core-derived-region, and said second portion may comprise CH2 and CH3 domains of said Ig protein. For example, said second portion may be directly linked to said 071 core-derived-region, and said second portion may comprise CH3 and CH4 domains of said Ig protein.

For one example of the protein, wherein said linker may be a peptide linker.

For one example of the protein, wherein said 071 core-derived-region may be linked directly or indirectly to the N-terminus of said second portion.

For one example of the protein, comprising the amino acid sequence as set forth in SEQ ID NO: 02, SEQ ID NO: 03, SEQ ID NO: 04, SEQ ID NO: 05, SEQ ID NO: 30 or SEQ ID NO: 31.

For one example of the protein, which may be a fusion protein.

For one example of the 071-core-fragment or the protein, which may be glycosylated.

For one example of the 071-core-fragment or the protein, which may be capable of binding to one or more Siglecs.

For one example of the 071-core-fragment or the protein, wherein said one or more Siglecs may comprise human Siglec.

For one example of the 071-core-fragment or the protein, wherein said one or more Siglecs may comprise Siglec-10.

For one example of the 071-core-fragment or the protein, which may be capable of binding to High Mobility Group Protein B1 (HMGB1).

For one example of the 071-core-fragment or the protein, wherein said 071 core may be derived from human protein. For example, said 071 core from other mammalian species are also provided.

The present disclosure provides an immunoconjugate, comprising the 071-core-fragment of the present disclosure, or the protein of the present disclosure.

The present disclosure provides a nucleic acid, encoding the 071-core-fragment of the present disclosure, or the protein of the present disclosure.

The present disclosure provides a vector, comprising the nucleic acid of the present disclosure.

The present disclosure provides a cell, comprising and/or expressing the 071-core-fragment of the present disclosure, the protein of the present disclosure, the immunoconjugate of the present disclosure, the nucleic acid of the present disclosure, and/or the vector of the present disclosure.

The present disclosure provides a composition, comprising the 071-core-fragment of the present disclosure, the protein of the present disclosure, the immunoconjugate of the present disclosure, the nucleic acid of the present disclosure, the vector of the present disclosure, and/or the cell of the present disclosure, and optionally a pharmaceutically acceptable carrier.

The present disclosure provides a method for preparing the 071-core-fragment of the present disclosure, or the protein of the present disclosure, comprising culturing the cell of the present disclosure under a condition enabling the expression of said 071-core-fragment or said protein.

The present disclosure provides a method for regulating a Siglec related signaling, comprising administering to a subject in need thereof an effective amount of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For example, the Siglec related signaling may comprise Siglec-mediated regulation of immune cell function. For example, the Siglec related signaling may comprise CD24-Siglec 10/G interaction. The method of the present disclosure, which may activate the Siglec related signaling. The method of the present disclosure, which may inhibit the Siglec related signaling.

The present disclosure provides the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in regulating a Siglec related signaling. For example, the Siglec related signaling may comprise Siglec-mediated regulation of immune cell function. For example, the Siglec related signaling may comprise CD24-Siglec 10/G interaction. For example, activating the Siglec related signaling. For example, inhibiting the Siglec related signaling.

The present disclosure provides a use of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for regulating a Siglec related signaling. For example, the Siglec related signaling may comprise Siglec-mediated regulation of immune cell function. For example, the Siglec related signaling may comprise CD24-Siglec 10/G interaction. For example, activating the Siglec related signaling. For example, inhibiting the Siglec related signaling.

The present disclosure provides a method for regulating an immune response, comprising administering to a subject in need thereof an effective amount of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure.

The present disclosure provides the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in regulating an immune response.

The present disclosure provides a use of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for regulating an immune response.

The present disclosure provides a method for repressing an immune-mediated tissue damage mediated by danger-associated molecular patterns (DAMPs), comprising administering to a subject in need thereof an effective amount of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in repressing an immune-mediated tissue damage mediated by danger-associated molecular patterns (DAMPs). For example, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides a use of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for repressing an immune-mediated tissue damage mediated by danger-associated molecular patterns (DAMPs). For example, wherein said immune-mediated tissue damage may be selected from the group consisting of graft vs host diseases, immunotherapy-related adverse events, rheumatoid arthritis, inflammatory bowel diseases (IBD), and multiple sclerosis (MS).

The present disclosure provides a method for preventing, ameliorating and/or treating a disease or condition caused by an inflammatory response arising from tissue injuries from infectious agents, comprising administering to a subject in need thereof an effective amount of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said disease or condition may be associated with viral infection. For one example of the method, wherein said disease or condition may be COVID-19. For one example of the method, wherein said disease or condition may be influenza. For one example of the method, wherein said disease or condition may be acquired immunodeficiency syndrome (AIDS). For one example of the method, wherein said disease or condition may be associated with bacterial infection. For one example of the method, wherein said disease or condition may be bacterial pneumonia.

The present disclosure provides the protein of the present disclosure, the immunoconjugate of the present disclosure, the nucleic acid of the present disclosure, the vector of the present disclosure, the cell of the present disclosure, and/or the composition of the present disclosure, for use in preventing, ameliorating and/or treating a disease or condition caused by an inflammatory response arising from tissue injuries from infectious agents. For example, wherein said disease or condition may be associated with viral infection. For example, wherein said disease or condition may be COVID-19. For example, wherein said disease or condition may be influenza. For example, wherein said disease or condition may be acquired immunodeficiency syndrome (AIDS). For example, wherein said disease or condition may be associated with bacterial infection. For example, wherein said disease or condition may be bacterial pneumonia.

The present disclosure provides a use of the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for preventing, ameliorating and/or treating a disease or condition caused by an inflammatory response arising from tissue injuries from infectious agents. For example, wherein said disease or condition may be associated with viral infection. For example, wherein said disease or condition may be COVID-19. For example, wherein said disease or condition may be influenza. For example, wherein said disease or condition may be acquired immunodeficiency syndrome (AIDS). For example, wherein said disease or condition may be associated with bacterial infection. For example, wherein said disease or condition may be bacterial pneumonia.

The present disclosure provides a method for preventing, ameliorating and/or treating a disease or condition caused by acute tissue damage from wound, comprising administering to a subject in need thereof an effective amount of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure. For one example of the method, wherein said disease or condition may be septicemia, crush syndrome and/or ischemia reperfusion injury.

The present disclosure provides the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure, for use in preventing, ameliorating and/or treating a disease or condition caused by acute tissue damage from wound. For example, wherein said disease or condition may be septicemia, crush syndrome and/or ischemia reperfusion injury.

The present disclosure provides a use of the 071-core-fragment of the present disclosure, the protein of the present disclosure, the immunoconjugate of the present disclosure, the nucleic acid of the present disclosure, the vector of the present disclosure, the cell of the present disclosure, and/or the composition of the present disclosure in the preparation of a medicament, wherein said medicament is used for preventing, ameliorating and/or treating a disease or condition caused by acute tissue damage from wound. For example, wherein said disease or condition may be septicemia, crush syndrome and/or ischemia reperfusion injury.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Generation of AI-071 Fc-Fusion Protein with Multiple Copies of 071 Core

To create DNA constructs encoding 071 Fc-fusion proteins, DNA encoding the peptide of either SEQ ID NO: 01, 02, 03, 04 or 05 can be fused with DNA encoding a signal peptide such as the signal peptide from human CD24 (SEQ ID NO: 32) at the N-terminal and DNA encoding human IgG1-hinge-CH2-CH3 region (SEQ ID NO: 28) at the C-terminal. These DNA can be synthesized in-vitro and cloned into expression plasmid vector such as pDNA3.1 by using stand recombinant DNA techniques. The recombinant plasmids can be transferred into mammalian cells such as CHO cells or human HEK-293T (293) cells by electroporation or other transfection methods. After electroporation or transfection, cells are cultured in serum-free media for 6-7 days and supernatants are then collected, passed over a column of Protein A resin such as MabSelect from GE Healthcare at a concentration not exceeding 16 g/L of resin (based on ELISA). The Fc-fusion protein bound on the column can be eluted and collected by using low pH solution such as 0.1 M acetic acid or citric acid (pH 3.5). Eluted protein can be re-suspended in PBS (pH=7.4) or other suitable buffers.

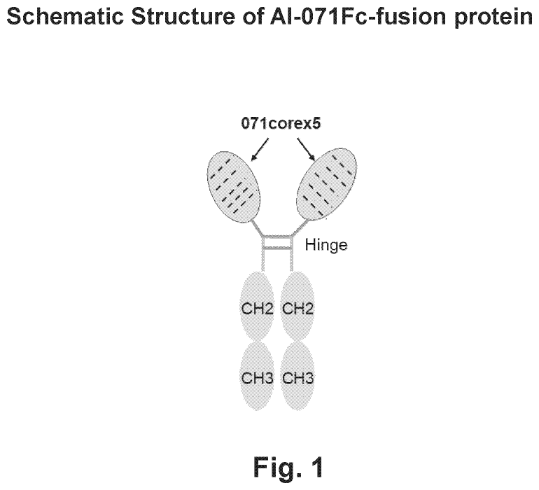

As an example, FIG. 1 shows the schematic structure of AI-071 fusion protein (hereafter also just called as AI-071), which was produced and secreted by cells transfecting with an expression plasmid vector with the insertion of DNA fragment encoding 5 copies of 071 core tandem repeats fused with a DNA fragment encoding human IgG1-Fc (hinge region, CH2 and CH3). As serving a biological control agent, human IgG1-Fc fusion protein without 071 core or other fragment was also produced by transfecting CHO cells with an expression plasmid vector with the insertion of DNA fragment encoding a peptide with amino acid sequences as showing in the SEQ ID NO: 29.

### Example 2 Characterization of AI-071 Fc-Fusion Protein

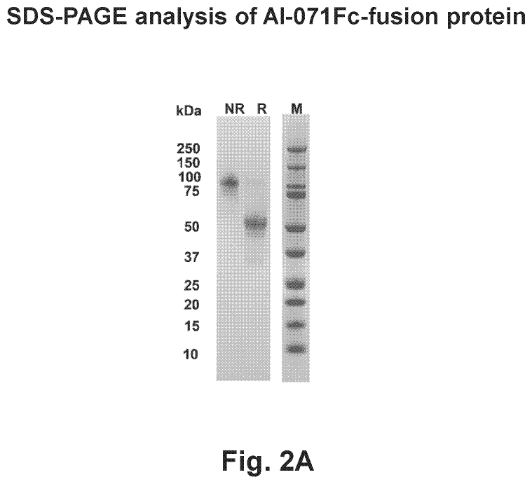

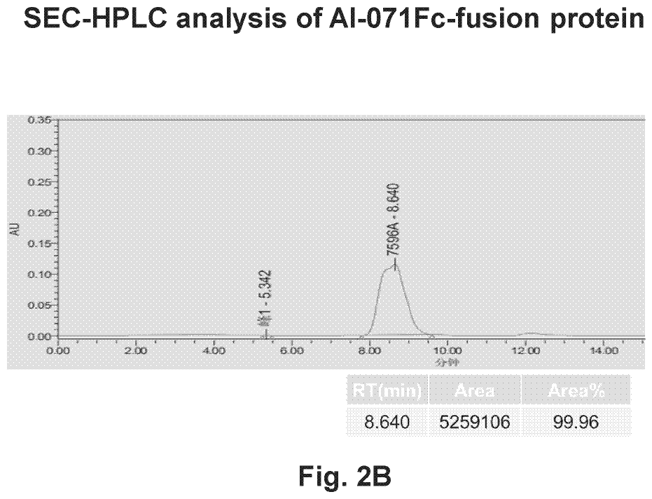

Approximately 2 μg of purified AI-071 protein, either in DTT-reducing or non-reducing conditions were loaded into SDS-PAGE gel. As shown in FIG. 2A, after electrophoresis, the size of this fusion protein sample in DTT-reduced condition is about the half of that in non-reducing condition, this agrees with the natural dimer formation of Fc-fusion protein. In either DTT-reducing or non-reducing conditions, the appearing molecule weight of AI-071 Fc-fusion protein is much greater than that deducted by protein peptide sequence only. For instance, the monomer of AI-071 mature peptide has 292 amino acid residues, which would have a predicted of protein molecule weight of about 31 kD in DTT-reducing gel. The additional gained molecule weight (about 20-25 kD, which contributes almost half of the molecule weight) detected in DTT-reducing gel is most likely from glycans attached to this glycoprotein.

The purified, intact AI-071 protein was further subjected to a SEC-HPLC analysis (FIG. 2B). As shown in FIG. 2B, a main peak with a retention time (RT) at 8.64 min and an area of 99.96% was detected by this SEC-HPLC analysis.

### Example 3 071 Core is Sialylated in AI-071

ELISA Analysis of AI-071 Protein with Different Anti-CD24 mAbs

The 071-core peptide is 12 amino acid long (SEQ ID NO: 1) and is derived from human CD24 molecule. The AI-071 protein, as illustrated in FIG. 1, comprising 5 copies of this 071-core peptide. The binding of AI-071 to different anti-CD24 mAbs can be measured by an enzyme linked immunosorbent assay (ELISA). The glycosylation and/or sialylation of the 071 core can be recognized by glycosylation and/or sialylation-dependent mAbs such as SN3 (ab134375 from Abcam), while the hypoglycosylation/unsialylated core can be recognized by H3L3 (a humanized anti-CD24 mAb, as described in the U.S. patent application No. 20210214458). The total amount of 071 core can be measured by the binding to another mouse mAb ML5, which recognizes human CD24 core peptide and is glycosylation and/or sialylation-independent.

For this purpose, an antigen direct binding ELISA was developed. Briefly, 96-well plates were coated with 10 μg/ml of either AI-071 (produced in CHO cells), CD24Fc (produced in CHO cells), CD24Fc (AcroBiosystems, Catalog #CD4-H5254, produced in human HEK293 cells) or human IgG1-Fc control (produced in CHO cells) at 4° C. overnight. After blocking with PBS-0.1% Tween20 solution (PBST), 100 μl of two-fold serial dilutions of either SN3 (ab134375 from Abcam), ML5 (ab278509 from Abcam) mouse mAb or humanized H3L3 mAb were added into the plates. The bound mouse SN3 or ML5 antibodies were detected by biotin-labeled goat anti-mouse IgG-Fc followed by HRP-labeled Avidin, whereas the bound humanized H3L3 antibody was detected by HRP-labeled goat anti-human IgG-Fab specific antibodies. The plates were then incubated with o-Phenylenediamine (OPD) substrates. After a color development at room temp for 15 mins, 1N HCl stop solution was added into the plates. The OD values at a wavelength of 492 nm (OD492) in each well were then measured.

The representative ELISA results are showed in FIGS. 2A-C, and the EC50 values are summarized in the table 1.

TABLE 1
Summary data of EC₅₀

| Detecting mAb | | | |
|---|---|---|---|
| (EC₅₀) | AI-071 | CD24Fc (CHO) | CD24Fc (HEK293) |
| SN3 (EC₅₀, M) | 1.310E−9 | No binding | 4.675E−9 |
| H3L3 | 3.150E−9 | 3.035E−9 | 2.848E−9 |
| (EC₅₀, M) | | | |
| ML5 (EC₅₀, M) | 6.922E−10 | 1.923E−9 | 1.430E−9 |

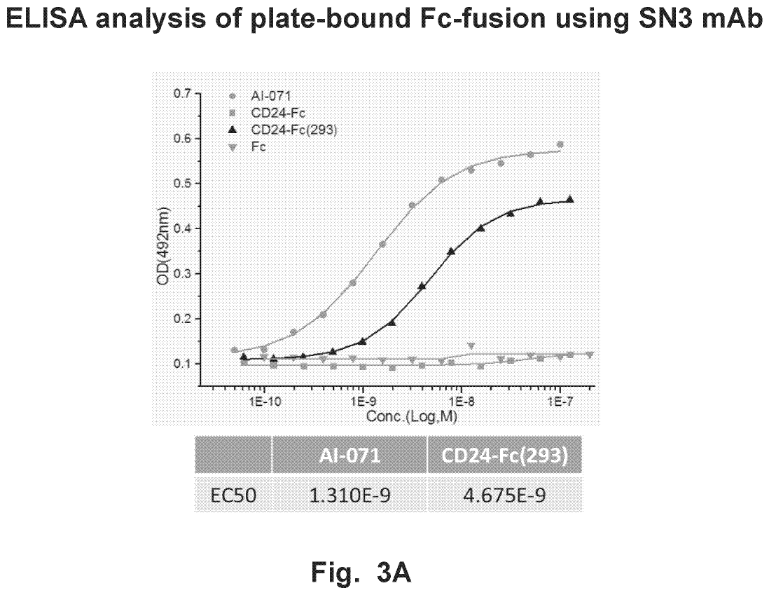

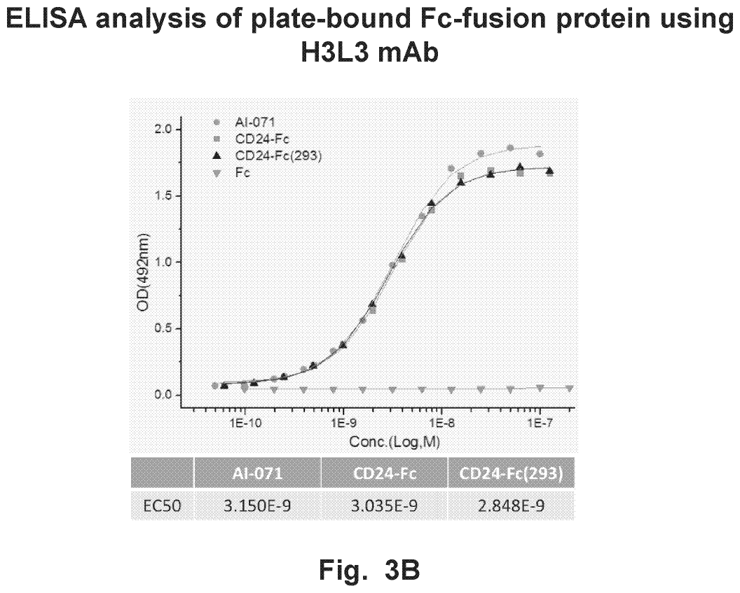

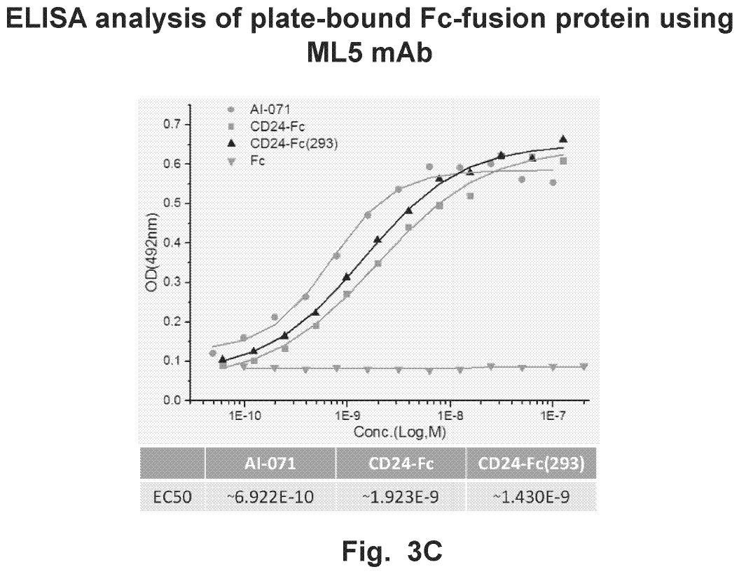

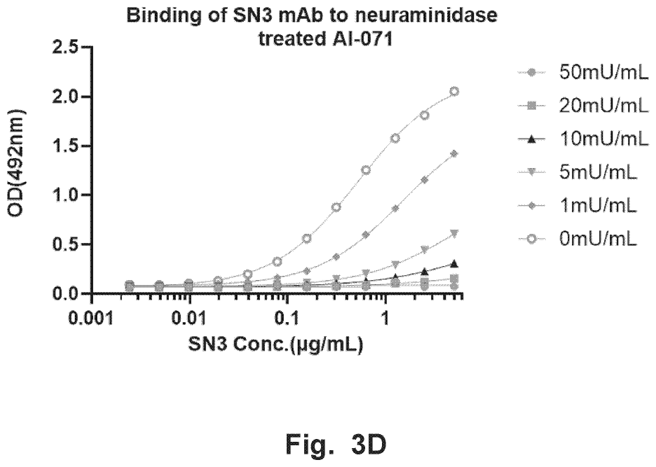

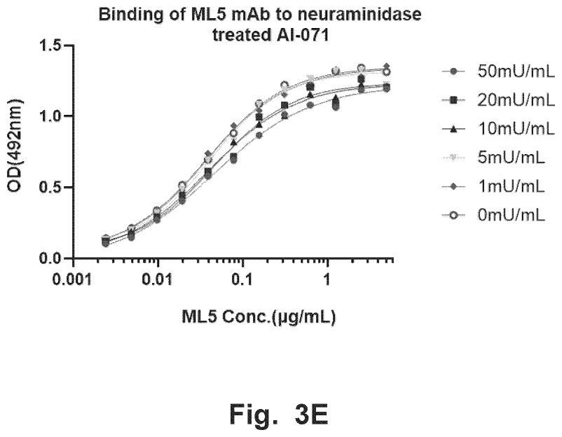

FIG. 3A shows the binding of AI-071 or CD24Fc to the glycosylation and/or sialylation dependent mAb SN3. As shown in the Figure, both CHO-derived AI-071 and HEK293-derived CD24Fc strongly bind to SN3, indicated that the core peptide is highly glycosylated and/or sialylated. However, CHO cell derived CD24Fc showed no binding at all.

H3L3 mAb binds to un-sialylated CD24 molecule (seen in the U.S. Patent Application 20210214458) and is thus a good indication for the extent of un-sialylated 071 core. As shown in FIG. 3C, and in Table 1, although AI-071 and CHO cells derived CD24Fc both bind to H3L3 at comparable EC50, AI-71 may contain less un-sialylated residues per 071 cores, as AI-071 has five copies of 071 core with a much more potent binding to ML5, which shows all antibody-accessible 071 peptide epitope.

To further verify the sialylation nature of AI-071 protein, AI-071 protein was treated with increasing concentration of (0, 1, 5, 10, 20 and 50 mU/mL) sialidases (also called as Neuraminidase, Sigma, Cat. No. N2876), which would remove and release terminal sialic acids from various glycomolecules). Sialidase-treated AI-071 protein was then coated in 96-well plate and probed for the binding to SN3 or ML5 mAb using the same ELISA. As shown in FIGS. 3D and 3E, sialidase (neuraminidase) treated AI-071 lost the binding to the sialylation-dependent SN3 mAb in a neuraminidase dose-dependent manner whereas the binding to sialylation-independent ML5 mAb was not affected. Thus, these results further demonstrate that AI-071 protein is sialylated.

As outlined above, the ability of these three mAbs binding to AI-071 allow the practitioner with ordinary skill to optimize the composition for increasing glycosylation and/or sialylation. For instance, one may generate constructs or culture conditions to increase the ratio of SN3/ML5 binding while decreasing the ratio of H3L3/ML5 binding, using the method disclosed herein, or other methods to measure antibody-antigen-binding.

In one embodiment, optimal sialyation vs total CD24 epitope, one may choose 1 copy to 10 copies of the 071 core to achieve optimal sialylation using the principle disclosed above.

In another embodiment, one may replace the amino acids within the 071 core, either uses homologous amino acids from other CD24 species from chimpanzee, monkey, dog, pig, mouse rat and etc. In yet another embodiment, one may switch the position of serine (Ser or S) or threonine (Thr or T) or increase or alter the pattern of amino acids to increase O-linked glycosylation using known art in the field. In yet another embodiment, one may increase sialyation of the 071 core using culture conditions that favors sialylation. In yet another embodiment, one may modify the CHO cells genetically to increase sialyotransferase activity.

### Example 4. Generation of Siglec Super Agonist Using Sialylated 071 Core

Defective Siglec function exacerbates inflammation caused by tissues injuries. Diseases associated with such inflammation includes both the classic sterile inflammation such as drug-induced liver damage, rheumatoid arthritis, inflammatory bowel diseases (IBD), multiple sclerosis, and the pathological settings in which infections cause tissue injuries such as COVID-19, influenza pneumonia and sepsis.

A super-agonist that shows enhanced and broad binding to multiple Siglecs may have a great valuation for treating diseases arising from inflammation caused by tissue injuries or infection.

### AI-071 has a Superior Binding to Siglec 10

To probe the interaction of AI-071 protein with the members of Siglec family such as Siglec10, an ELSIA assay was developed. In-brief, 96-well plates were coated (100 μL per well) with 0.2 μg/mL of HEK293 cell derived Siglec10-mlgG2aFc fusion protein (AcroBiosystems, SIO-H525b) at 4° C. overnight. After washing and blocking with SuperBlock (Thermo, 37515) at room temperature for 1 hour, 100 μL of 2-fold serial dilutions of either AI-71 or CD24Fc (all starting at 1.5 mg/ml) were added. The bound AI-071 or CD24Fc protein was then detected by HRP-labeled goat anti-human IgG-Fc antibody (1:5000, Invitrogen, A18829) followed by the addition of Tetramethylbenzidin (TMB) substrates. After a color development at room temp for 15 min, 2N HCl stop solution was added into the plate. The OD values at a wavelength of 450 nm (OD 450 nm) in each well were then measured.

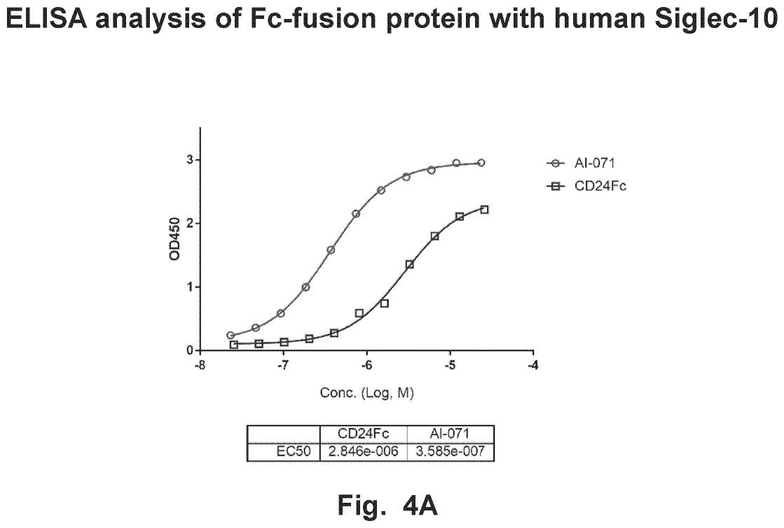

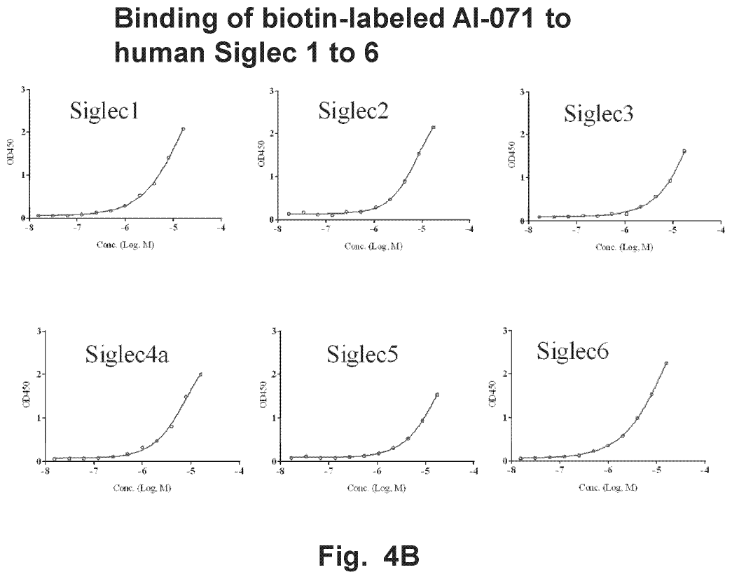

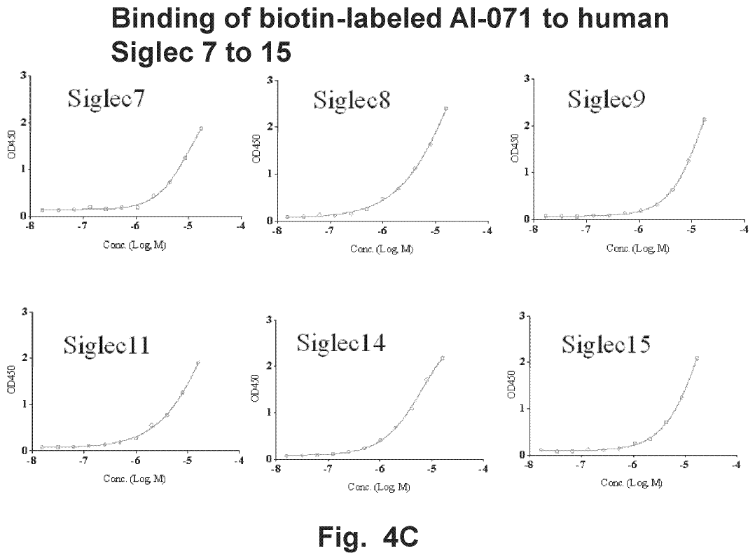

FIG. 4A shows one of the representative ELISA results. As shown in the Fig, AI-071 has a much higher binding (EC₅₀ of 3.585E-7M) to Siglec10 than CD24Fc has (EC₅₀: 2.846E-6M). Thus, this data indeed demonstrated that AI-071 has a superior binding to Siglec10.

To evaluate the spectrum of Siglecs that binds to AI-071, recombinant IgG-Fc fusion protein containing one of the human siglec family members (Siglec-1, 2, 3, 4a, 5, 6, 7, 8, 9, 11, 14 and 15) were coated in 96-well plates and their binding to biotin-labeled AI-071 protein was detected in ELISA. The representative ELISA results are shown in FIG. 4B (Siglec-1, 2, 3, 4a, 5 and 6) and FIG. 4C (Siglec-7, 8, 9, 11, 14 and 15). As shown in Figs., AI-071 showed cross-reactivity to Siglects 1-6, 7-15. These results indicate a broad cross-reactivity of AI-071 to multiple human Siglecs.

### Binding Kinetics of AI-07 Fusion Protein to Siglec10

A Fortebio based biolayer interferometry assay (BLI) was used to further analyze the binding kinetics of AI-07 fusion protein to human Siglec10. For this propose, streptavidin-coated biosensor tubes (Sartoris Octet SA biosensors, Cat. 18-0009) were incubated with biotinylated human Siglec10 (AcroBiosystems, HEK293 cell derived, His- and Avi-tagged, SIO-H82E3) at 2 μg/mL in binding buffer (1×PBS with 1 mM MgCl2, 3 mM CaCl2) at room temperature for 10 mins. After balanced with binding buffer, analytes of different concentrations of AI-071 or human IgG-Fc control protein were loaded into individual biosensor tube and the binding kinetic curves (association and dis-association) were recorded in Fortebio-BLITZ instrument.

Results for this experiment are summarised in Table 2. In this system, both the AI-071 and human IgG1-Fc control samples showed Siglec10 binding. But, clearly data in table 2 showed that AI-071 has more than 10-fold higher Siglec10 binding affinity than control Fc protein has.

TABLE 2
Binding Kinetics of AI-071 and Human
IgG Fc Protein to Human Siglec10

| Samples | Ka (M⁻¹ · S⁻¹) | kd (S⁻¹) | K_{D} (M) |
|---|---|---|---|
| AI-071 | 4.162E3 | 1.248E−4 | 2.997E−8 |
| (lot: 20210811) | | | |
| Human Fc-control | 2.262E3 | 9.475E−4 | 4.189E−7 |
| (lot: 20210805) | | | |

Abbreviations:
Ka = Binding association constant;
Kd = dissociation constant;
KD = Affinity constant

### Example 5 AI-071 has a Superior Binding to High Mobility Group Box 1 (HMGB1)

To demonstrate that AI-071 protein also binds to HMGB1, a similar ELISA assay was developed. In-brief, 96-well plates were coated (100 μL per well) with 0.1 μg/mL HMGB1-His tag protein (AcroBiosystems, HM1-H5220, HEK293 cell derived) at 4° C. overnight. After blocking with SuperBlock (Thermo, 37515) at room temperature for 1 hour, 100 μL of 2-fold serial dilutions of AI-71 or CD24Fc (all starting at 1.5 mg/ml, and diluted in PBST-1% BSA solution containing 1 mM MgCl2 and 1 mM CaCl2) were added. The bound AI-071 or CD24Fc protein was then detected by adding HRP-labeled goat anti-human IgG-Fc antibody (1:1000, Invitrogen, A18829), followed by the addition of tetramethylbenzidin (TMB) substrates. After a color development at room temp for 15 mins, stop solution (2N HCl) was then added into the plate. The OD values at a wavelength of 450 nm in each well were then measured.

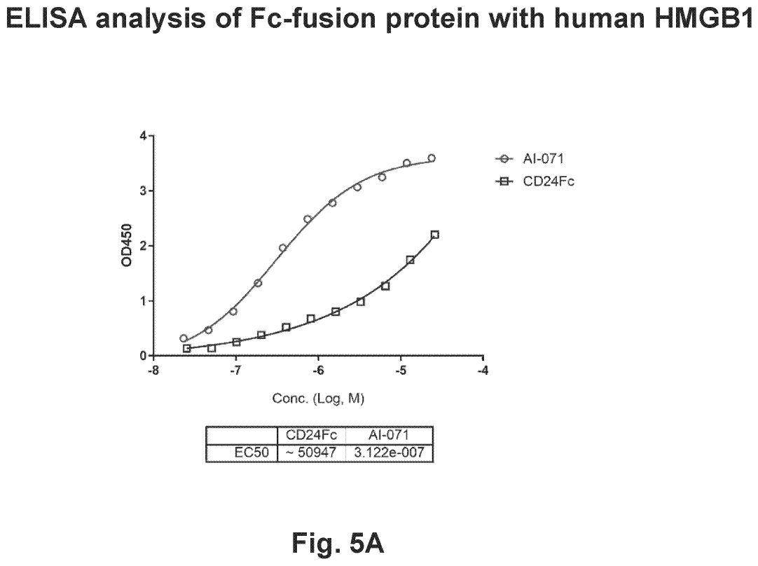

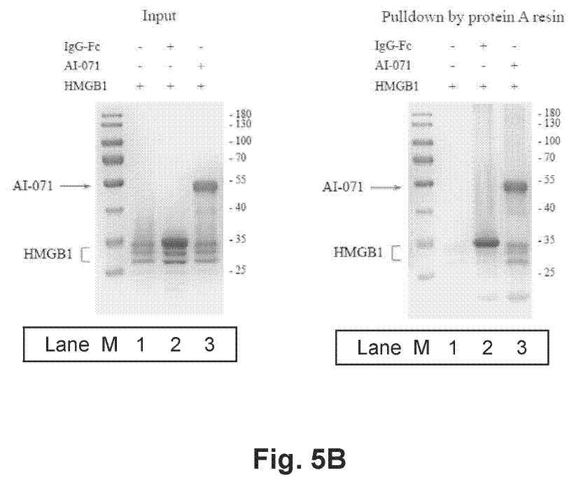

FIG. 5A shows the representative ELISA results. As shown in Figure, AI-071 has a more than 50-fold higher HMGB1-binding activity (EC₅₀: 3.122E-7M) than CD24Fc has (EC50: 5.095E-5M). Thus, this data demonstrated that AI-071 also has a superior binding to HMGB1.

### Probe of AI-071 and HMGB1 Interaction by Pull-Down Assay

To further verify the superior binding of AI-071 to HMGB1, a protein pull-down assay was developed (FIG. 5B). In-brief, 8 μL of 500 μg/mL HMGB1-His protein was mixed with either 5.7 μL of 700 μg/mL AI-071, 10 μL of 250 μg/mL human IgG1-Fc control protein or none, and set at room temp for 5 min. The mixtures were then incubated with protein A-conjugated beads to capture (or pull-down) the bound proteins. The captured proteins were separated in SDS-PAGE gel and visualized by Coomassie Brilliant Blue dye staining.

FIG. 5B shows one of the representative HMGB1 pull-down assay results. As shown in Fig, HMGB1 proteins are clearly pull-down (captured) by AI-071, but not by IgG1-Fc control sample. Thus, this dada further demonstrated that AI-071 has superior HMGB1-binding activities.

### Example 6 AI-071 Protect Mice Against Lethal IBD

Dextran sulfate sodium (DSS)-induced inflammatory bowel diseases (IBD) in mice

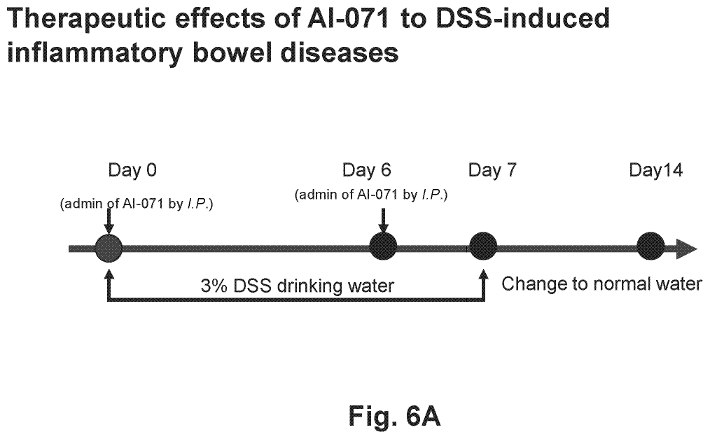

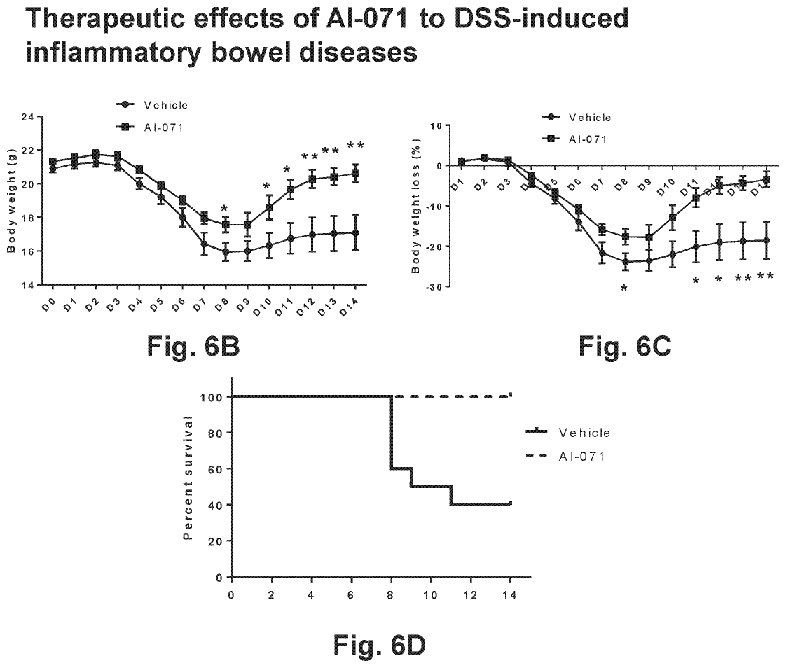

The DSS-induced IBD model is shown in FIG. 6A and the testing results are shown in FIGS. 6B, 6C and 6D. As shown in FIG. 6A and the following table (table 3), C57BL/6N female mice (6-8 weeks old, average weight about 21 gram) were fed with 3% DSS in the drinking water for 7 days, and were monitored daily, for weight loss, disease progression and survival. On day 0, mice were randomly divided into two groups (10 animals/group): one group was administered with AI-071 fusion protein by i.p. injection (dose: 50 mg/kg or about 1 mg) on day 0 and day 6, the other group was administered with vehicle control (0.9% NaCl) by same i.p. injection.

TABLE 3

| Treatment group (N) | Modeling | Dosage (mg/kg) | Dosing Frequency |
|---|---|---|---|
| AI-071 (n = 10) | DSS | 50 | Day 0 and Day 6 |
| Vehicle (n = 10) | DSS | vehicle | Day 0 and Day 6 |

On day 7, DSS water was removed and mice were then fed with normal drinking water and continue to be monitored daily, for recovery and survival, up to day 14.

The colitis progression was measured by the Disease Activity Index (DAI), and scored as in the following table.

TABLE 4
Disease Activity Index (DAI) parameters

| Score | Weight loss | Stool consistency | Bleeding |
|---|---|---|---|
| 0 | No loss | Normal | No blood |
| 1 | 1-5% | Mild soft | Brown color |
| 2 | 5-10% | Very soft | Reddish color |
| 3 | 10-20% | Diarrhea | Bloody stool |
| 4 | >20% | | Gross bleeding |

DAI is obtained by the sum of each individual score.

In our experimental setting, animals in each group started to show signs of disease and weight loss (FIGS. 6B and 6C) from days 3 to 5 after drinking 3% DSS water. However, compared to those in the saline vehicle control treatment group, animals in AI-071 treatment group showed a faster recovery in body weight gain (in grams, FIG. 6B) or low body weight lose rate (%, FIG. 6C) after switched to the normal drinking water on day 7. The mean difference in the body weight between AI-071 treatment group and the vehicle treatment group is either statistically significant on day 8 and day 10 (P<0.05 by two-tailed T-test, marked * on the top of AI-071 group in the FIG. 6B), or statistically very significant on days 12, 13 and 14 (p<0.01 by two-tailed T-test, marked ** on the top of AI-071 group in the FIG. 6B). Similarly to that, as shown in FIG. 6C, the mean difference in the body weight lose rate between AI-071 treatment group and vehicle treatment group is also either statistically significant on days 8, 11 and 12 (P<0.05 by two-tailed T-test, marked as * under the vehicle group), or statistically very significant on day 13 and day 14 (p<0.01 by two-tailed T-test, marked as ** under the vehicle group). Moreover, in the vehicle treatment group, 4 of 10 mice (40%) were found dead as early as day 8, and by day 11, a total of 6 mice (60%) were dead, thus survive rate in this group at day 14 drop to 40% (FIG. 6D). In contrast, in the AI-071 protein treatment group, no death occurred and therefore a 100% survive rate was archived, which is statistically very significant (P=0.0038, Log-rank test). These data clearly demonstrated that administration of AI-071 protein can protect subjects against lethal IBD, such as DSS-induced colitis in mice.

### Example 7, Effect of AI-071 on LPS-Induced Acute Airway/Lung Inflammation in Mice

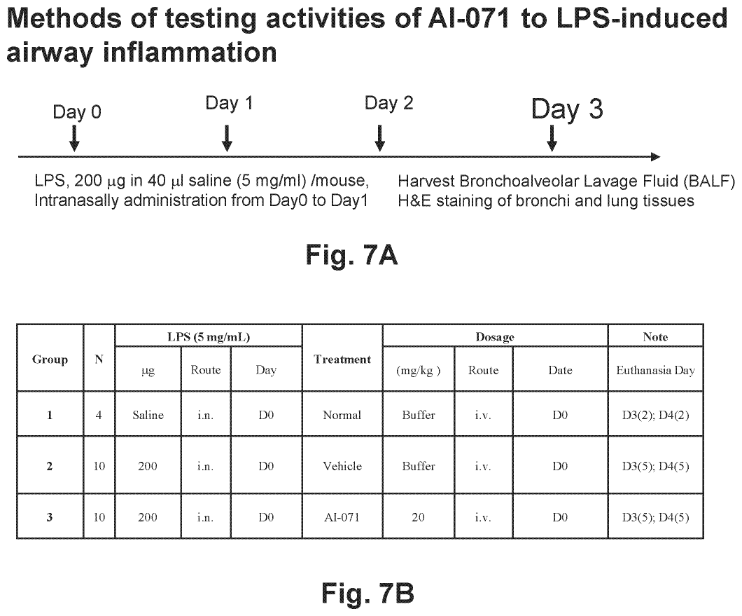

Lipopolysaccharides (LPS) induced acute lung disease in mice model was developed here and used for testing the therapeutic effect of AI-071 protein to airway/lung inflammation (FIG. 7A). Briefly, 200 μg LPS in 40 μl saline was administered into mouse by intranasally on day 0, and day 1. On Day 3 or day 4, mice were sacrificed and bronchoalveolar lavage fluids (BALF) were harvested and the total cell account, neutrophil cell number, level of cytokines and AI-071 proteins in BALF were counted or measured; meanwhile, the lung tissues were also collected and processed for haematoxylin and eosin (H&E) staining.

As shown in FIG. 7B and the following table.

TABLE 5

| | | | | | | | | | Note: |
|---|---|---|---|---|---|---|---|---|---|
| | | LPS (5 mg/mL) | | | | Dosage | | | Euthanasia |
| Group | N | μg | Route | Day | Treatment | mg/kg | Route | Date | Day |
| 1 | 4 | Saline | i.n. | D 0 | Normal | Buffer | I.V. | D 0 | D 3(2); |
| | | | | | | | | | D 4(2) |
| 2 | 10 | 200 | i.n. | D 0 | Vehicle | Buffer | I.V. | D 0 | D 3(5), |
| | | | | | | | | | D 4(5) |
| 3 | 10 | 200 | i.n. | Do | AI-071 | 20 | I.V. | D 0 | D 3(5); |
| | | | | | | | | | D 4(5) |

On day 0, mice were randomly divided into different treatment groups (each group has 10 animals) and were administered once by i.v. injection on day 0 with either vehicle control (0.9% NaCl saline, Group 1 and group 2) or AI-071 fusion protein (Group 3).

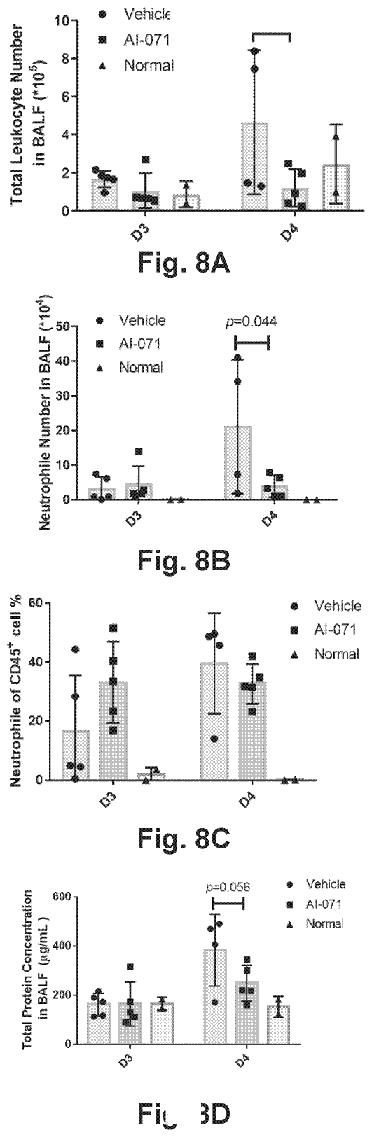

The results were in FIG. 8A-8D. As shown in FIG. 8C, A single IV dose of AI-071 (20 mg/kg) attenuated LPS-induced acute airway inflammation which was observed as a decrease in the number of leukocytes, neutrophils and total amount of proteins present in BALF samples. Histopathological analysis and pathological lesion scoring of collected lung tissues also showed that LPS-stimulated mice treated with AI-071 had mild lung pathological damage (FIGS. 8C and 8D) compared to those in vehicle control treated group (FIGS. 8B and 8D).

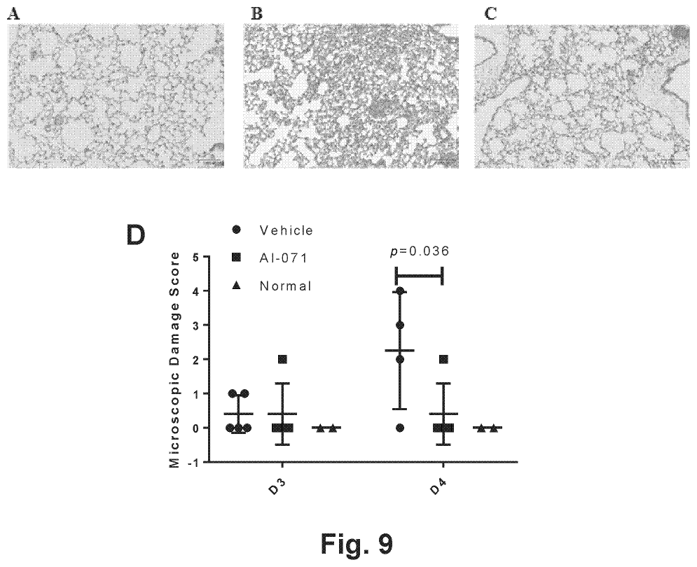

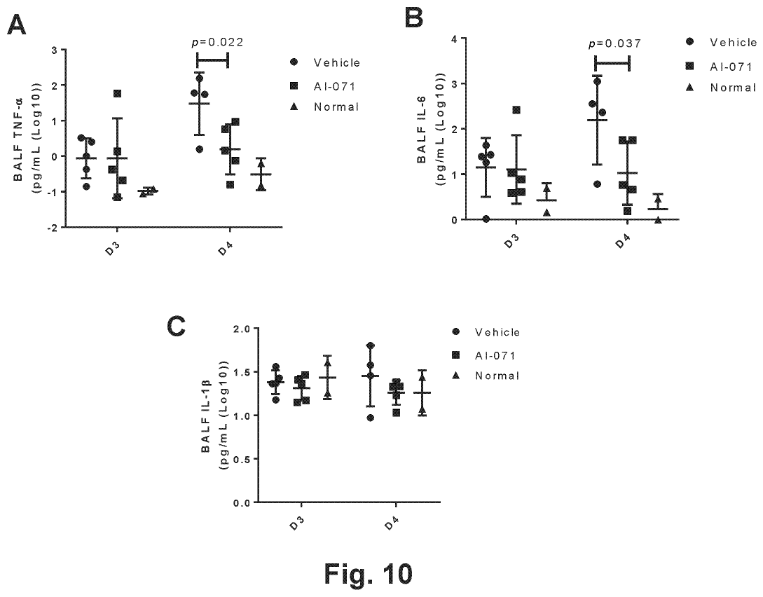

The levels of cytokines in BALF samples from normal mice or LPS stimulated mice treated with vehicle or AI-071 were also measured by using a LEGENDplex™ Mouse Inflammation Panel (13-plex) multi-analyte flow assay kit (BioLegend). This panel is a multiplex beads-based assay using fluorescence-encoded beads suitable for use on various flow cytometers. Using a total of 13 bead populations distinguished by size and internal fluorescent dye, this panel allows simultaneous quantification of 13 mouse cytokines (TNF-α, IL-la, IL-1B, IL-6, IL-10, IL-12p70, IL-17A, IL-23, IL-27, CCL2 (MCP-1), IFN-β, IFN-γ and GM-CSF). Most of cytokines in this panel are produced by innate immune cells, linking the innate and adaptive immunity and/or by stander cells. In this example, the samples were analyzed using a Cytek™ Northern Lights-CLC full spectrum flow-cytometer (Cytek Biosciences, Inc.). The typical results are shown in FIG. 9A-9D. As shown in FIG. 9A-9D, the levels of some pro-inflammatory cytokines such as TNF-α (FIG. 9A) and IL-6 (FIG. 9B) from LPS stimulated mice were increased in either day 3 or day 4 BALF samples compared to those in normal mice. More interestingly, in the LPS-stimulated mice, the levels of TNFα (FIG. 9A) and IL-6 (FIG. 9b) in BALF on day 4 from AI-071 treatment group were significantly reduced compared to those in the vehicle control treatment group, again demonstrating the efficacy of AI-071 on suppressing or inhibiting PAMPs (LPS) induced cytokine production or release. The levels of some other cytokines such as IL-1B (FIG. 9C) in day 3 BALF samples were almost same as those in day 4 BALF samples and also no much differences appeared between the normal mice and LPS stimulated mice either treated with the vehicle control or AI-071. The levels of other cytokines such as II.-10, IL-12p70, IL-17A and IL-23 in BALF samples were all below the detection limit and therefore not presented.

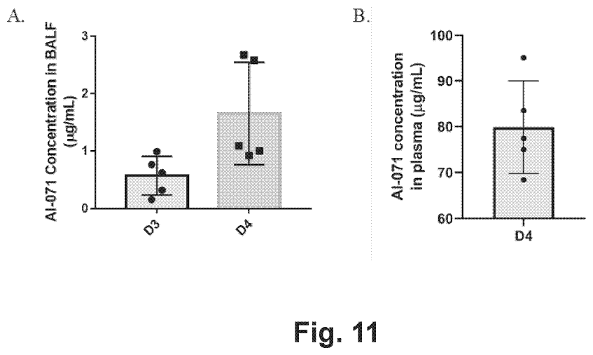

The amount of AI-071 protein present in either BALF or blood plasma samples from AI-071 treated mice was also measured using ELISA, and the results are shown in FIG. 11A-11B. The results in FIG. 11A show that the concentration of AI-017 in BALF increased between Day 3 and Day 4 after dosing which may explain why the efficacy was strongest on Day 4. The results also demonstrate that concentrations of AI-017 in BALF are much lower than concentrations achieved in blood plasma (FIG. 11B).

### Example 8 Diagram for Testing the Effect of AI-071 in Mouse Arthritis Model

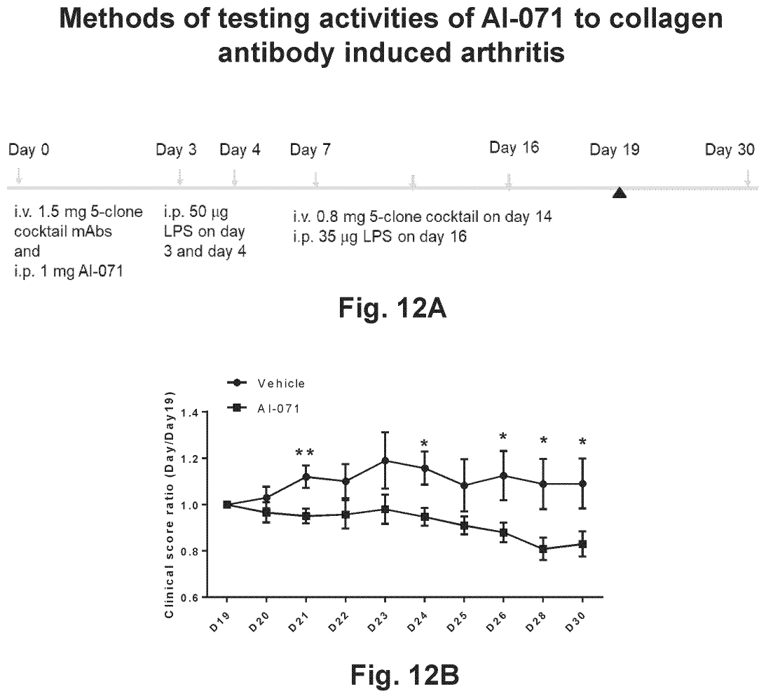

For this propose, a collagen antibody induced arthritis (CAIA) model was developed. The disease model and treatment schedule are shown in FIG. 12A. Briefly, 7-8 weeks old female Balb/c mice are administered (1.5 mg/mouse) a cocktail of 5 anti-collagen mAbs by i.v. injection on day 0, followed by i.p. injection of 50 μg LPS on day 3 and day 4. On day 0, mice were randomly divided into two different treatment groups (each group has 10 mice): one group was treated with AI-071 (50 mg/kg, i.v. injection), the other was treated with saline vehicle control. On day 14, these mice were re-administered with 0.8 mg of the anti-collagen mAb cocktail by i.v. injection, followed by i.p. injection of 35 μg LPS on day 16. On day 19, AI-071 treated mice were administrated with a second dose (1 mg) by i.p. injection, whereas control treated mice were administrated with saline by i.p. injection. All of these mice are monitored daily from day 0, up to day 30.

In our experimental setting, animals in each group started to show signs of disease and/or body weight loss from days 5 to day 7 after 1^{st} administration of anti-collagen mAb cocktail. However, as shown in the FIG. 12B, after an 2^{nd} administration of anti-collagen mAb cocktail on day 14, LPS on day 16, and AI-071 protein or saline vehicle control on day 19, the AI-071 protein treated group showed a reduced disease score ratio (day/day 19) from day 20 to day 30, whereas the vehicle control treated group showed no such reduction. The mean difference between the AI-071 treatment group and vehicle treatment group is either statistically very significant on day 21 (P<0.01 by two-tailed T-test, marked as ** on the top bar of vehicle group in the FIG. 12B), or statistically significant on days 24, 26, 28 and 30 (p<0.05 by two-tailed T-test, marked as * on the top bar of vehicle group in the FIG. 12B). Therefore, these data demonstrated that AI-071 protein might also have therapeutic value in the treatment of subjects with arthritis.

### Example 9 Generation of Different Versions of AI-071Fc-Fusion Protein Containing 5 Tandem Repeats of a 12 AA-Long Core Peptide Other than 071 Core

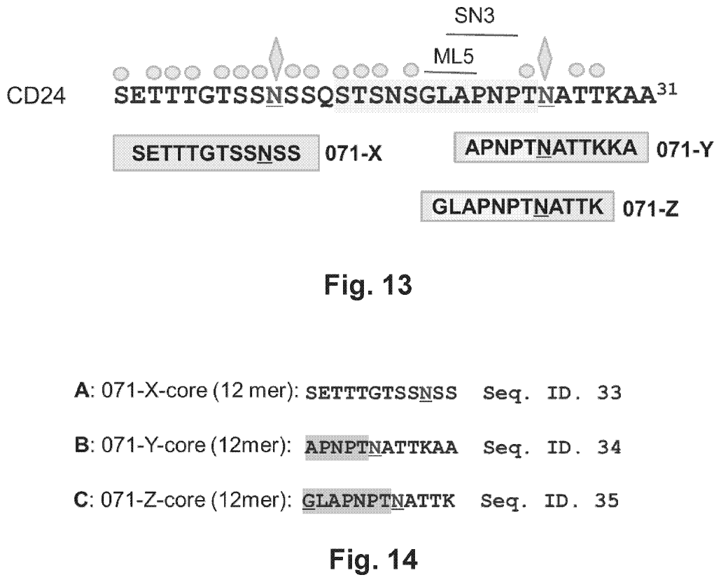

As shown in FIG. 13, although the peptide backbone of mature human CD24 molecule is only about 31-amino acid long, but it contains up to 16 potential O-linked glycosylation sites (Ser and Thr) and 2 potential N-linked glycosylation sites. The O-linked glycosylation sites are distributed along the whole peptide backbone regions including the 071core region, while the two N-linked glycosylation sites are flanked at each side of the 071core region. To test if peptides from these other regions of CD24 protein have any different biological activity, 3 different peptides (namely as 071-X, 071-Y and AI-07-Z, and as shown in FIG. 13, each is 12 amino acid-long) corresponding to the N-terminal or the C-terminal region of CD24 were selected and used for construction of a new series of AI-07-Fc-fusion proteins. There are 0 amino acid in 071-X (SEQ ID NO:33), 5 amino acids in 071-Y (SEQ ID NO:34) and 7 amino acids in 071-Z (SEQ ID NO:35) that are overlapped with the 12 AA-long 071core peptide (SEQ ID NO:01). However, as shown in FIGS. 13 and 14, each of these 3 peptides all contains a single N-linked glycosylation site and multiple O-lined glycosylation sites (9 in 071-X, 3 in both 071-Y and 071-Z). Similar to the construction of AI-071-Fc protein (which has 5 tandem repeats of 071 core peptide) described in example 1, 5-tandem repeats of each of these three peptides were also linked together and fused with the Fc-tail of human IgG1 (hinge region, CH2 and CH3), thus creating 3 different versions of Fc-fusion proteins (namely AI-071-X, AI-071-Y, and 071-Z). Briefly, DNA encoding the peptide of either SEQ ID NO:36, 37 or 38 was fused with DNA encoding the signal peptide from human CD24 (SEQ ID NO: 32) at the N-terminal and DNA encoding human IgG1-hinge-CH2-CH3 region (SEQ ID NO: 28) at the C-terminal. These DNA molecules were synthesized in-vitro and cloned into expression plasmid vector by using stand recombinant DNA techniques and the recombinant plasmids were transferred into CHO cells by electroporation. After electroporation, cells were cultured in serum-free media for 6-7 days and supernatants were then collected, passed over a column of Protein A resin (MabSelect from GE Healthcare). The Fc-fusion protein bound on the column were eluted and collected by using low pH solution such (0.1 M acetic acid, pH 3.5). All these three fusion proteins were successfully produced and purified from supernatants collected from CHO transfectants by protein A-column. The identity and quality of these fusion proteins were analyzed by SDS-PAGE and the results are described in detail in example 11.

### Example 10 Generation of AI-071-Fc Fusion Protein Variants Containing Either One Copy or Two Copies of 071 Core Peptide

Since the 12 amino-acid long 071 core peptide (STSNSGLAPNPT, SEQ ID NO: 01) contains 5 potential mucin-like O-linked glycosylation sites (serine or threonine in STP motif) and no N-linked glycosylation site, it is expected that adding just one repeat or two repeats of this core peptide to the Fc-tail of human IgG would be able to generate a Fc-fusion protein coming with the features of glycosylation and/or sialylation modifications when these proteins are produced in mammalian cells. To test if this is the case, Fc-fusion protein contains either one repeat (namely as AI-071-1copy) or two tandem repeats of 071 core peptide (namely as AI-071-2copies) were generated by using the similar method as shown in example 1. Briefly, DNA encoding the peptide of either SEQ ID NO: 01 or 02 was fused with DNA encoding the signal peptide from human CD24 (SEQ ID NO: 32) at the N-terminal and DNA encoding human IgG1-hinge-CH2-CH3 region (SEQ ID NO: 28) at the C-terminal. These DNA molecules were synthesized in-vitro and cloned into expression plasmid vector by using stand recombinant DNA techniques and the recombinant plasmids were transferred into CHO cells by electroporation. After electroporation, cells were cultured in serum-free media for 6-7 days and supernatants are then collected, passed over a column of Protein A resin (MabSelect from GE Healthcare). The Fc-fusion protein bound on the column were eluted and collected by using low pH solution such (0.1 M acetic acid, pH 3.5). These two fusion proteins were successfully produced and purified from CHO transfectants by protein A-column. The identity and quality of these two fusion proteins were analyzed by SDS-PAGE and their results are described in detail in example 11.

### Example 11 Characterization of the Different Versions of AI-07 Fusion Proteins by SDS-PAGE

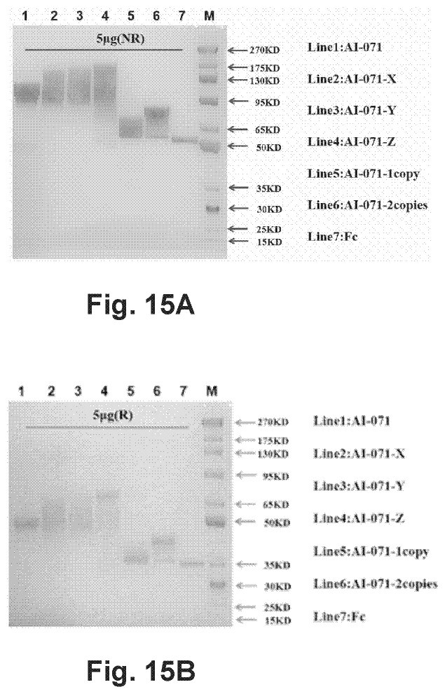

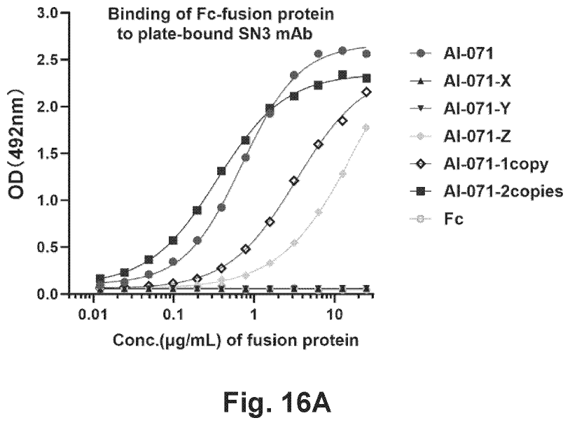

FIGS. 15A and 15B show one of the representative SDS-PAGE gel electrophoresis analysis results of the purified, different versions of AI-07-Fc protein (lanes 2 to 6: AI-071-X, AI-071-Y, AI-071-Z, AI-071-1copy and AI-071-2 copies), along with AI-071 (lane 1) and IgG-Fc (lane 7) served as control samples for a comparison. As shown in the FIG. 15A, under non-reducing conditions, the appear molecule weight of each of these 7 fusion proteins is about twice of that in reducing conditions (FIG. 15B), which is in-agreement with a disulfide-linked homodimer form of these fusion proteins in their nature conditions. Compared to the protein bands/pattern detected in AI-071 sample (lane 1), under either reducing or non-reducing conditions, the protein bands detected in AI-071X (lane 2), AI-071-Y (lane 3) or AI-071-Z (lane 4) samples all showed a heterogenous and smear pattern, with an appear molecule weight ranging from 100 to 150 kDa under non-reducing condition and 50-75 kDa under reducing conditions, all significantly larger than their predicted molecular weight of about 62 kDa band based on the homodimer amino acid sequence (584AA) or 31 kDa band based on the monomer amino acid sequence (292AA). The appear additional 40-90 kDa molecular mass gained in these 3 fusion proteins, which represents about 40-70% of whole molecule weight, is most likely contributed by the massive saccharides attached to the protein core-peptide backbone. The more heterogenous and larger smear pattern bands seen in these 3 fusion proteins may be due to the facts that these fusion proteins contain N-linked sites in addition to the multiple O-linked glycosylation sites in their tandem repeats. Thus, in general, the appear molecule weight of the main band detected in these three fusion molecules is either equal to or greater than that of AI-071 protein, an indication of extensively glycosylation modification.

Also in the figure, for the versions of AI-071-Fc fusion protein containing either one repeat of 071core (AI-071-1copy, lane 5) or two repeats of 071 core (AI-071-2copies, lane 6), under either reducing or non-reducing conditions, the appear molecule weight or the protein bands detected is smaller than AI-071 (lane 1) but larger than that in control IgG1-Fc (lane 7), which contains the Fc-tail portion only. The samples of AI-071-1copy or AI-071-2copies also showed a more heterogenous protein bands/patterns compared to IgG-Fc control sample. These results indicate that AI-071-Fc fusion protein variants containing either one repeat or two tandem repeats of the 12 AA-long 071 core peptide also come with some extent of glycosylation modification.

### Example 12 Further Characterization of the New Series of AI-07 Fusion Proteins: Probing their Interactions with Glycosylation and/or Sialylation-Dependent or Independent Antibodies in ELSIA

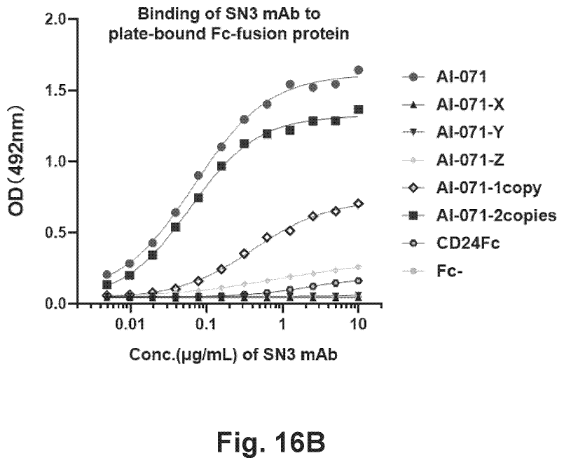

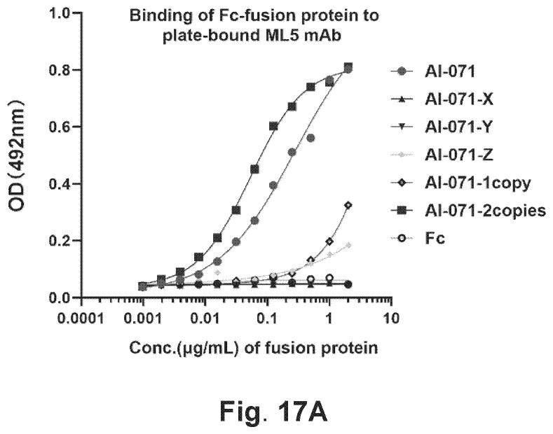

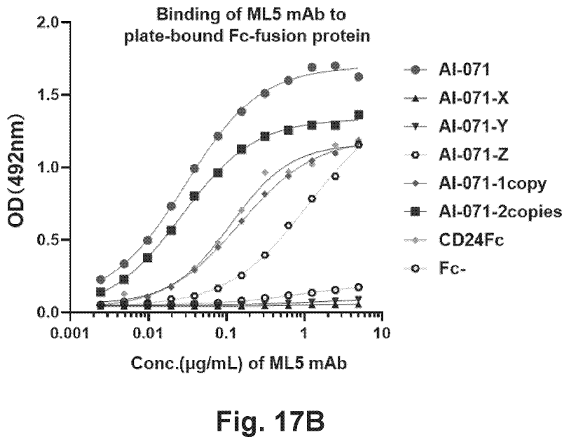

The binding of the above different versions of AI-071 Fc-fusion protein containing multiple tandem repeats of a 12 AA-long core peptide other than 071 core (AI-071-X, AI-071-Y and AI-071Z) or AI-071-Fc fusion protein variants containing either one repeat or two tandem repeats of 071 core peptide (AI-071-1copy, and AI-071-2copies) to glycosylation/sialylation-dependent SN3 mAb or glycosylation/sialylation-independent ML5 mAb were investigated by using the same ELISA methods as shown in example 3. The ELISA results are summarized in FIG. 16 and FIG. 17. FIGS. 16A and 16B show the results of SN3 mAb binding to different versions of AI-07-Fc protein or IgG-Fc control protein. Data from FIG. 16A show that samples from AI-071-1copy, AI-071-2 copies, AI-071-Z or AI-071 but not AI-071-X, AI071-Y or human IgG-Fc control sample have a dose-response binding to the SN3 mAb that pre-coated on the plate. The binding intensity in AI-071-2 copies sample is same to that seen in AI-071, while the binding intensity in AI-071-1 copy sample or AI-071Z sample is weaker than in AI-071. Similar results were seen (FIG. 16B) when a fixed concentration of the analyzed samples (i.e. AI-07-Fc series fusion proteins) were coated on the plate and 2-fold serial dilutions of SN3 mAb were added to the plate, except a much weak binding intensity was detected in AI-071Z sample. CHO cell derived CD24Fc shows no binding to SN3 in this assay, which is consistent with the result seen in example 3. The absence of the binding of SN3 mAb to AI-071-X or AI-071-Y is not unexpected, as shown in FIG. 13, the 071-X core and 071-Y core contains either no or only a portion of the amino acid sequences/epitopes recognized by SN3 mAb.

FIGS. 17A and 17B show the results of ML5 mA binding to different versions of AI-07-Fc protein or human IgG-Fc control protein with b. Data from FIG. 17A show that samples from AI-071-1 copy, AI-071-2 copies, AI-071-Z or AI-071 but not AI-071-X, AI071-Y or human IgG-Fc control sample have a dose-response binding to the ML5 mAb pre-coated on the plate. Again, the binding intensity in AI-071-2 copies sample is same to that seen in AI-071, while the binding intensity in AI-071-1copy sample or AI-071Z sample is much weaker than in AI-071. Again, similar results were seen when a fixed concentration of the analyzed samples (i.e. AI-07-Fc series fusion proteins) were coated on the plate and 2-fold serial dilutions of ML5 mAb were added to the plate (FIG. 17B). In consistent with the result seen in example 3, CHO cell derived CD24Fc also shows the binding to ML5 mAb in this assay. The ML5 binding intensity or the curve seen in AI-071-1copy sample is almost same as that seen in CD24Fc, which contains one copy of the peptide sequence/epitope recognized by ML5. The absence of ML5 binding to AI-071-X or AI-071-Y is not also unexpected, as 071-X core and 071-Y core contains either no or only a portion of the amino acid sequence/epitope recognized by ML5 mAb.

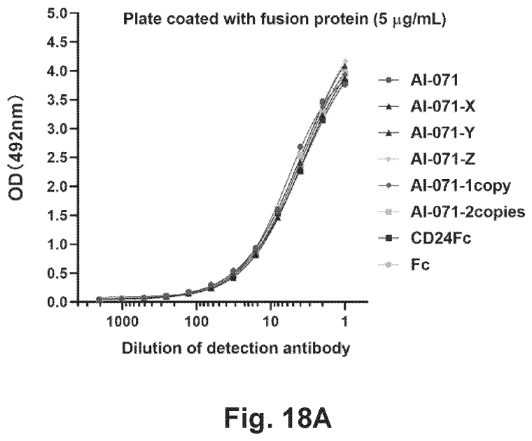

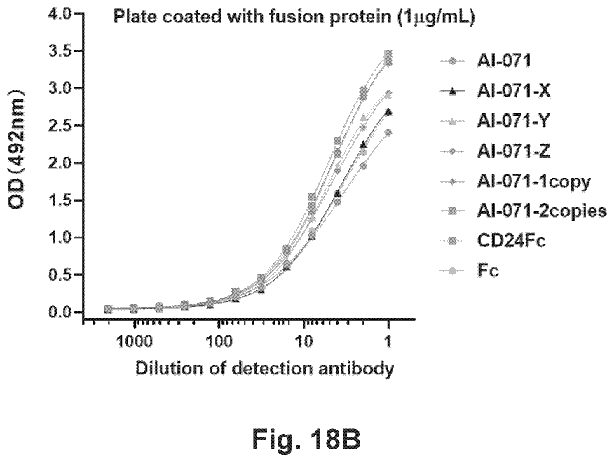

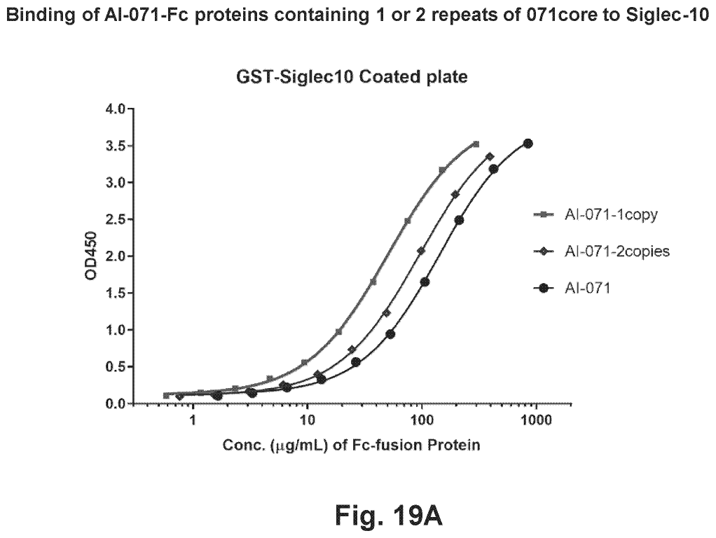

To rule-out the binding differences among different versions of AI-07 fusions protein seen in either SN3 or ML5 based ELSIA is due to different quality of prepared fusion proteins or plate coating variations, the same prepared protein samples were coated into the 96-well plates at either low (1 μg/mL) or high concentration (5 μg/mL) and the plate-bound proteins were then detected by adding 2-fold serial dilutions of an HRP-conjugated goat anti-human IgG-Fc antibody. The results were shown in FIGS. 18A and 18B. As shown in FIG. 18A, when a high concentration (5 μg/mL) of these proteins was coated in the plates, an essentially same dose-response binding curve was seen in all the samples tested, demonstrating the precision of this ELISA assay. A similar pattern was seen when a low concentration (1 μg/mL) of these proteins was coated in the plates (FIG. 18B).

Taken together, these data indicate that the binding differences among different versions of AI-07 fusions protein seen in SN3 or ML5 based ELSIA is not due to the differences in the quality of proteins or the plate coating variations.

### Example 13: Detection the Binding of AI-071-Fc Proteins Containing Either 1 Repeat or 2 Repeats of 071Core to Human Siglec-10 or HMGB1

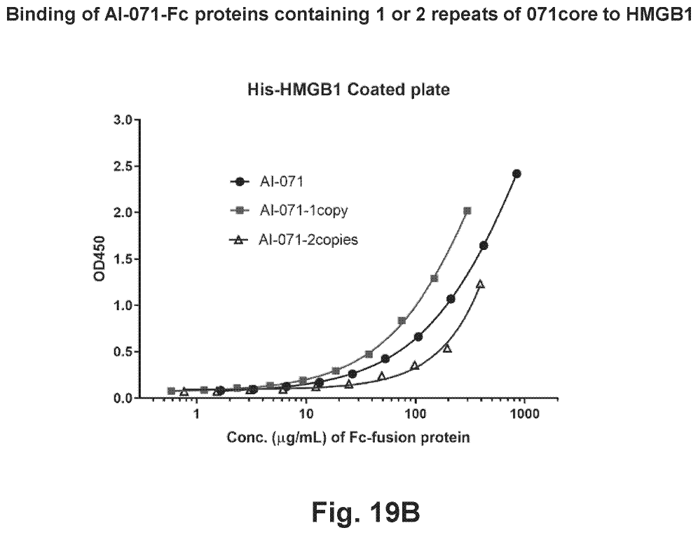

To test if the AI-071-Fc protein variants containing either 1 repeat or 2 repeats of 071core also interact with HMGB1 and Siglecs such as Siglec-10, a similar ELISA assay method as seen in the example 5 (Siglec-10 binding ELISA) or in the example 6 (HMGB1 binding ELISA) was used. The representative ELISA results are shown in FIGS. 19A and 19B. As shown in FIG. 19A, like that in AI-071, AI-071-Fc proteins containing either 1 copy (AI-071-1copy) or 2 copies of 071core (AI-071-2copies) both have same in-vitro binding activity to Siglec-10 molecule. Similarly, as shown in FIG. 19B, like that in AI-071, both AI-071-1copy sample or AI-071-2copies sample showed the in-vitro binding to HMGB1. These results indicate that adding just one repeat or two tandem repeats of the 12 AA-long 071core to the IgG-Fc tail would be able to generate glycoproteins capable of binding to Siglec-10 or HMGB1.

### Example 14: Detection the Binding of AI-071-Fc Protein Variants Containing 5 Tandem Repeats of a 12-AA Long Peptide Other than 071cor Peptide to Human Siglec-10 or HMGB1

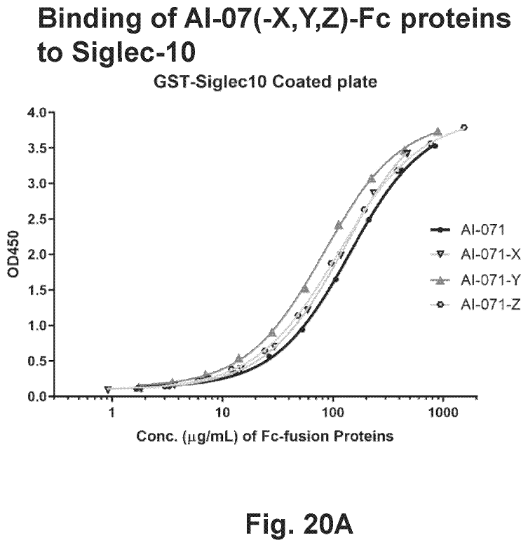

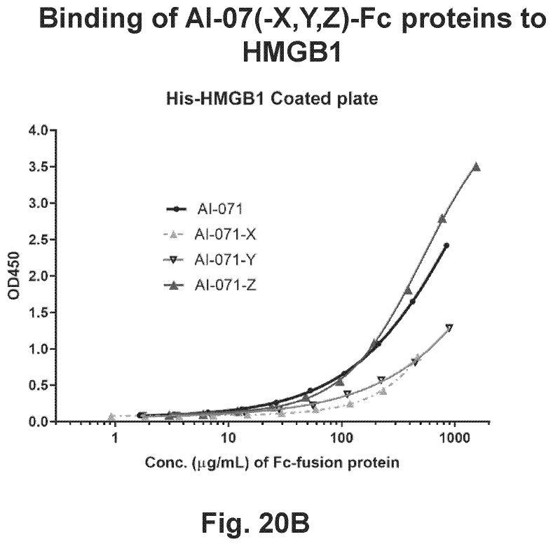

To test if these versions of AI-071-Fc protein also interact with HMGB1 or Siglecs such as Siglec-10, again a similar ELISA assay method as seen in the example 5 (Siglec-10 binding ELISA) or seen in the example 6 (HMGB1 binding ELISA) was used. The representative results are shown in FIGS. 20A and 20B. As shown in FIG. 20A, AI-071-Fc variant proteins containing 5 tandem repeats of a 12-AA long peptide other than 071 cor peptide (AI-071-X, AI-071-Y and AI-071 Z) or AI-071 all showed the binding to human Siglec-10 in this assay. When these AI-071-Fc variant proteins were tested for the binding to HMGB1, different results were observed. As shown in FIG. 20B, among three AI-071-Fc variant proteins tested, only the AI-071Z protein sample showed a comparable binding to HMGB1 as that of AI-071. The other two variant proteins (AI-071X and AI-071Y) showed a much weaker binding to HMGB1. These results showed that a simply linking of 5 repeats of a 12 AA-long peptide other than 071core into IgG-Fc may not be able to fully recapitulate the function the 071 core peptide, an indication of the specificity or unique feature of the 071 core.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the afore mentioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An isolated 071-core-fragment, wherein an amino acid sequence of said isolated 071-core-fragment is as set forth in SEQ ID NO: 01.

2. A protein, comprising:
a 071 core-derived-region,
wherein said 071 core-derived-region consists of:
a single copy of a 071-core-fragment; or
two or more copies of 071-core-fragments directly or indirectly linked to each other;
wherein an amino acid sequence of said 071-core-fragment is as set forth in SEQ ID NO: 01, and
wherein the protein does not comprise a fragment derived from a CD24 other than the 071-core-fragment.

3. The protein of claim 2, comprising 2-5 of said two or more copies of 071-core-fragments.

4. The protein of claim 2, wherein at least two of said two or more copies of 071-core-fragments are directly linked to each other.

5-6. (canceled)

7. The protein of claim 2, wherein said 071 core-derived-region consists of the amino acid sequence as set forth in SEQ ID NO: 01, SEQ ID NO: 06, SEQ ID NO: 07, SEQ ID NO: 08, SEQ ID NO: 09, SEQ ID NO: 10, SEQ ID NO: 11, SEQ ID NO: 12, SEQ ID NO:13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21.

8. The protein of claim 2, further comprising a second portion,
wherein said second portion comprises a half-life extending portion.

9-10. (canceled)

11. The protein of claim 2, further comprising a second portion,
wherein said second portion comprises an immunoglobulin fragment.

12. The protein of claim 11, wherein said immunoglobulin fragment comprises a Fc portion of immunoglobulin.

13. The protein of claim 11, wherein said immunoglobulin fragment comprises a hinge region of immunoglobulin.

14. The protein of claim 11, wherein said immunoglobulin fragment comprises a CH2 domain.

15. The protein of claim 11, wherein said immunoglobulin fragment comprises a CH3 domain.

16. (canceled)

17. The protein of claim 11, wherein an immunoglobulin is selected from the group consisting of IgG1, IgG2, IgG3, IgG4, IgM and IgA.

18-20. (canceled)

21. The protein of claim 11, wherein said 071 core-derived-region is linked directly or indirectly to an N-terminus of said second portion.

22. The protein of claim 2, comprising the amino acid sequence as set forth in SEQ ID NO: 02, SEQ ID NO: 03, SEQ ID NO: 04, SEQ ID NO: 05, SEQ ID NO: 30 or SEQ ID NO: 31.

23. (canceled)

24. The protein of claim 2, wherein the protein is glycosylated.

25. The protein of claim 2, wherein the protein is capable of binding to one or more Siglecs.

26.-27. (canceled)

28. The protein of claim 2, wherein the protein is capable of binding to High Mobility Group Protein B1 (HMGB1).

29-39. (canceled)

40. A method for repressing an immune-mediated tissue damage mediated by danger-associated molecular patterns (DAMPs), comprising:
administering to a subject in need thereof an effective amount of the protein of claim 2.

41. (canceled)

42. A method for preventing, ameliorating and/or treating a disease or condition caused by an inflammatory response arising from tissue injuries from infectious agents, comprising:
administering to a subject in need thereof an effective amount of the protein of claim 2.

43-48. (canceled)

49. A method for preventing, ameliorating and/or treating a disease or condition caused by acute tissue damage from wound, comprising:
administering to a subject in need thereof an effective amount of the protein of claim 2.

50. (canceled)


SEQUENCE LISTING (38 sequences)

SEQ ID NO: 1 - protein, 12 aa, synthetic construct
  REGION 1..12: note = 071core
STSNSGLAPNPT

SEQ ID NO: 2 - protein, 24 aa, synthetic construct
  REGION 1..24: note = 071core 2x
STSNSGLAPNPTSTSNSGLAPNPT

SEQ ID NO: 3 - protein, 36 aa, synthetic construct
  REGION 1..36: note = 071core 3x
STSNSGLAPNPTSTSNSGLAPNPTSTSNSGLAPNPT

SEQ ID NO: 4 - protein, 48 aa, synthetic construct
  REGION 1..48: note = 071core 4x
STSNSGLAPNPTSTSNSGLAPNPTSTSNSGLAPNPTSTSNSGLAPNPT

SEQ ID NO: 5 - protein, 60 aa, synthetic construct
  REGION 1..60: note = 071core 5x
STSNSGLAPNPTSTSNSGLAPNPTSTSNSGLAPNPTSTSNSGLAPNPTSTSNSGLAPNPT

SEQ ID NO: 6 - protein, 12 aa, synthetic construct
  REGION 1..12: note = 071core-V1
SASNSGLAPNPT

SEQ ID NO: 7 - protein, 12 aa, synthetic construct
  REGION 1..12: note = 071core -V2
STSNSGLAPNPA

SEQ ID NO: 8 - protein, 12 aa, synthetic construct
  REGION 1..12: note = 071core -V3
SASNSGLAPNPA

SEQ ID NO: 9 - protein, 12 aa, synthetic construct
  REGION 1..12: note = 071core -V4
  SITE 1: note = misc_feature - Xaa = Ser or Thr
XTSNSGLAPNPT

SEQ ID NO: 10 - protein, 12 aa, synthetic construct
  REGION 1..12: note = 071core -V5
  SITE 2: note = misc_feature - Xaa = Ser or Thr
SXSNSGLAPNPT

SEQ ID NO: 11 - protein, 12 aa, synthetic construct
  REGION 1..12: note = 071core -V6
  SITE 3: note = misc_feature - Xaa = Ser or Thr
STXNSGLAPNPT

SEQ ID NO: 12 - protein, 12 aa, synthetic construct
  REGION 1..12: note = 071core -V7
  SITE 5: note = misc_feature - Xaa = Ser or Thr
STSNXGLAPNPT

SEQ ID NO: 13 - protein, 12 aa, synthetic construct
  REGION 1..12: note = 071core -V8
  SITE 12: note = misc_feature - Xaa = Ser or Thr
STSNSGLAPNPX

SEQ ID NO: 14 - protein, 12 aa, synthetic construct
  REGION 1..12: note = 071core -V9
  SITE 1: note = misc_feature - Xaa = Ser or Thr
  SITE 12: note = misc_feature - Xaa = Ser or Thr
XTSNSGLAPNPX

SEQ ID NO: 15 - protein, 12 aa, synthetic construct
  REGION 1..12: note = 071core -V10
  SITE 5: note = misc_feature - Xaa = Ser or Thr
  SITE 6: note = misc_feature - Xaa = Ser or Thr
STNSXXAPNPPT

SEQ ID NO: 16 - protein, 12 aa, synthetic construct
  REGION 1..12: note = 071core -V11
STSNSGFAPNPT

SEQ ID NO: 17 - protein, 12 aa, synthetic construct
  REGION 1..12: note = 071core -V12
SSQSTSAAPSPA

SEQ ID NO: 18 - protein, 12 aa, synthetic construct
  REGION 1..12: note = 071core -V13
SSQNTSTTPNPA

SEQ ID NO: 19 - protein, 12 aa, synthetic construct
  REGION 1..12: note = 071core -V14
GNQNISASPNPT

SEQ ID NO: 20 - protein, 12 aa, synthetic construct
  REGION 1..12: note = 071core -V15
GNQSISAAPNPT

SEQ ID NO: 21 - protein, 12 aa, synthetic construct
  REGION 1..12: note = 071core -V16
SSQSTSTAPNPA

SEQ ID NO: 22 - protein, 18 aa, synthetic construct
  REGION 1..18: note = hinge 1
EPKSCDKTHTCPPCPAPE

SEQ ID NO: 23 - protein, 18 aa, synthetic construct
  REGION 1..18: note = hinge 2
EPKSSDKTHTCPPCPAPE

SEQ ID NO: 24 - protein, 18 aa, synthetic construct
  REGION 1..18: note = hinge 3
EPKSSDKTHTSPPSPAPE

SEQ ID NO: 25 - protein, 13 aa, synthetic construct
  REGION 1..13: note = hinge 4
DKTHTCPPCPAPE

SEQ ID NO: 26 - protein, 16 aa, synthetic construct
  REGION 1..16: note = IgG4hinge
ESKYGPPCPPCPAPAE

SEQ ID NO: 27 - protein, 18 aa, synthetic construct
  REGION 1..18: note = IgA1hinge
VPSTPPTSPSTPPTPSPS

SEQ ID NO: 28 - protein, 232 aa, synthetic construct
  REGION 1..232: note = human IgG1 hingeCH2CH3 region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 260 aa, synthetic construct
  REGION 1..260: note = human IgG1Fc protein
MGRAMVARLGLGLLLLALLLPTQIYSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 292 aa, synthetic construct
  REGION 1..292: note = Fc-fusion protein with five repeats of the 071core
STSNSGLAPNPTSTSNSGLAPNPTSTSNSGLAPNPTSTSNSGLAPNPTSTSNSGLAPNPTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 318 aa, synthetic construct
  REGION 1..318: note = signal peptide+Fc-fusion protein with five repeats of the 071core
MGRAMVARLGLGLLLLALLLPTQIYSSTSNSGLAPNPTSTSNSGLAPNPTSTSNSGLAPNPTSTSNSGLAPNPTSTSNSGLAPNPTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 26 aa, synthetic construct
  REGION 1..26: note = signal peptide
MGRAMVARLGLGLLLLALLLPTQIYS

SEQ ID NO: 33 - protein, 12 aa, synthetic construct
  REGION 1..12: note = 07x-core
SETTTGTSSNSS

SEQ ID NO: 34 - protein, 12 aa, synthetic construct
  REGION 1..12: note = 07y-core
APNPTNATTKAA

SEQ ID NO: 35 - protein, 12 aa, synthetic construct
  REGION 1..12: note = 07z-core
GLAPNPTNATTK

SEQ ID NO: 36 - protein, 60 aa, synthetic construct
  REGION 1..60: note = 07x-core5x
SETTTGTSSNSSSETTTGTSSNSSSETTTGTSSNSSSETTTGTSSNSSSETTTGTSSNSS

SEQ ID NO: 37 - protein, 60 aa, synthetic construct
  REGION 1..60: note = 07y-core5x
APNPTNATTKAAAPNPTNATTKAAAPNPTNATTKAAAPNPTNATTKAAAPNPTNATTKAA

SEQ ID NO: 38 - protein, 60 aa, synthetic construct
  REGION 1..60: note = 07z-core5x
GLAPNPTNATTKGLAPNPTNATTKGLAPNPTNATTKGLAPNPTNATTKGLAPNPTNATTK